(19) United States
(12) Patent Application Publication
(10) Pub. No.: US 2024/0041994 A1
(43) Pub. Date: Feb. 8, 2024

(54) CMV VACCINE AND METHOD OF MAKING AND USING THE SAME

(71) Applicants: The Regents of the University of California, Oakland, CA (US); The Board of Regents of the University of Oklahoma, Norman, OK (US)

(72) Inventors: Lewis L. LANIER, San Francisco, CA (US); William H. HILDEBRAND, Oklahoma City, OK (US)

(21) Appl. No.: 18/311,557

(22) Filed: May 3, 2023

Related U.S. Application Data
(63) Continuation of application No. 16/616,435, filed on Nov. 22, 2019, now abandoned.
(60) Provisional application No. 62/510,546, filed on May 24, 2017.

(51) Int. Cl. A61K 39/00; A61K 39/395; A61K 39/12; C07K 14/74

(52) U.S. Cl. CPC A61K 39/0005; A61K 39/3955; A61K 39/12; C07K 14/70539; A61K 2039/505; A61K 2039/572; A61K 2039/58

(57) ABSTRACT

The present invention provides vaccine compositions for preventing and/or treating cytomegalovirus (CMV) infection and methods of making and using the same.

### CROSS REFERENCE TO RELATED APPLICATION

This application claims the benefit of priority to U.S. Provisional Patent Application No. 62/510,546 filed May 24, 2017, the entire contents of which are incorporated by reference herein and for all purposes.

### STATEMENT REGARDING FEDERALLY SPONSORED RESEARCH OR DEVELOPMENT

This invention was made with government support under grant no. R21 AI108860 awarded by the National Institutes of Health. The government has certain rights in the invention.

### FIELD

The present disclosure relates, inter alia, to vaccine compositions for preventing and/or treating cytomegalovirus (CMV) and methods of making and using the same.

### BACKGROUND

Cytomegalovirus (CMV) infects half of the population in the United States, establishing a lifelong persistent infection. CMV is particularly dangerous for infants born with this infection, which occurs in 20,000-40,000 infants born each year in the United States. Twenty percent of these infected babies develop permanent disabilities such as microcephaly, hearing loss, vision impairment, and learning disabilities. CMV is the leading cause of non-genetic deafness in children and approximately 400 children per year die annually. In addition, CMV is life-threatening for individuals with a compromised immune system, including solid organ and hematopoietic stem cell transplant patients. Anti-viral drugs have significant side effects and there is no vaccine.

### SUMMARY

The disclosure herewith provides methods and compositions inducing an immune response in a subject in need thereof. In particular, the subject may need an immune response to treat and/or prevent CMV infection.

In one aspect, the disclosure provides a composition containing at least one HLA-E ligand or a fragment, derivative or variant thereof capable of binding to HLA-E and a pharmaceutically acceptable carrier, wherein the composition is capable of inducing an immune response.

In embodiments, in the composition the HLA-E ligand is capable of binding to a) a CD94-NKG2C receptor that is present on natural killer (NK) cells or T cells; or b) a T cell antigen receptor.

In embodiments, in the composition the HLA-E ligand binds with high affinity to a) a CD94-NKG2C receptor that is present on NK cells or T cells; or b) a T cell antigen receptor.

In embodiments, in the composition the HLA-E ligand is associated with HLA-E when binding to a) a CD94-NKG2C receptor that is present on NK cells or T cells; or b) a T cell antigen receptor.

In embodiments, the composition can further contain a linker.

In embodiments, the composition can contain more than one HLA-E ligand or a fragment, derivative or variant thereof.

In embodiments, the composition can further contain HLA-E or a fragment, derivative or variant thereof.

In embodiments, in the composition the HLA-E or the fragment, derivative or variant thereof can be a soluble and secretory form.

In embodiments, in the composition the immune response can include an increase of NK cell-mediated killing or T cell-mediated killing of cytomegalovirus (CMV)-infected cells.

In embodiments, in the composition the immune response can include proliferation of T cells and/or NK cells.

In embodiments, in the composition the T cells and/or NK cells may express CD94-NKG2C receptor.

In embodiments, in the composition the T cells and/or NK cells may be specific for CMV-infected cells.

In embodiments, in the composition the immune response can include increase of T cells and/or NK cells that express CD94-NKG2C receptor.

In embodiments, in the composition the immune response can include killing of CMV-infected cells.

In embodiments, in the composition the immune response can include T cell-mediated killing of CMV-infected cells.

In embodiments, in the composition the immune response can include NK cell-mediated killing of CMV-infected cells.

In embodiments, in the composition the immune response can be increased production or secretion of one or more cytokines.

In embodiments, in the composition the cytokine can be interferon gamma.

In embodiments, in the composition the HLA-E or the fragment, derivative or variant of HLA-E and the HLA-E ligand or the fragment, derivative or variant of HLA-E ligand may not substantially bind to inhibitory CD94-NKG2A receptor of NK cells or T cells.

In embodiments, the composition can be formulated as a CMV vaccine.

In embodiments, in the composition the HLA-E ligand or the fragment, derivative or variant thereof can be selected from the group consisting of the sequences identified in Table 1 and Table 2 and sequences having at least 85% identity to the sequences identified in Table 1 and Table 2. In embodiments, in the composition the HLA-E ligand or the fragment, derivative or variant thereof can be selected from the group consisting of the sequences as set forth in SEQ ID NOs: 4-129 and sequences having at least 85% identity to the sequences as set forth in SEQ ID NOs: 4-129.

In embodiments, in the composition the HLA-E or the fragment, derivative or variant thereof can contain the sequence of SEQ ID NO. 1, 2, or 3, or a variant thereof having at least 85% identity to the sequence of SEQ ID NO. 1, 2, or 3.

In embodiments, in the composition the HLA-E or the fragment, derivative or variant of HLA-E and the HLA-E ligand or the fragment, derivative or variant of HLA-E ligand can be covalently associated via a linker.

In embodiments, in the composition the linker can contain the sequence of a (G₄S)₃, (G₄S)₄ or (G₄S)₅.

In another aspect, the present disclosure provides a method of inducing an immune response in a subject. The method can include administering, to a subject, an effective amount of a composition comprising at least one HLA-E ligand or a fragment, derivative or variant thereof capable of binding to HLA-E and a pharmaceutically acceptable carrier.

In embodiments, in the method the subject can have or be suspected of having CMV infection.

In embodiments, in the method the subject may not have CMV infection.

In embodiments, in the method the subject may be a child or an infant.

In embodiments, in the method the subject may be a woman prior to pregnancy.

In embodiments, in the method the subject may have a compromised immune system.

In embodiments, in the method the subject may be a transplant patient.

In embodiments, in the method the induction of immune response can include expansion of NK cells or T cells.

In embodiments, in the method the induction of immune response can include an increase of cells expressing NKG2C in the subject.

In embodiments, in the method the cells expressing NKG2C can be NK cells and/or T cells.

In embodiments, in the method the viral load of CMV can be decreased.

In still another aspect, the present disclosure provides a method of making a composition inducing an immune response. The method can include formulating at least one HLA-E ligand or a fragment, derivative or variant thereof capable of binding to HLA-E and a pharmaceutically acceptable carrier in a form suitable for administration.

In embodiments, in the method the HLA-E or a fragment, derivative or variant of HLA-E can be formulated with the HLA-E ligand or the fragment, derivative or variant of HLA-E ligand capable of binding to HLA-E and the pharmaceutically acceptable carrier in a form suitable for administration.

In still another aspect, the present disclosure provides a method of making a composition for inducing an immune response. The method can include introducing a vector sequence encoding a recombinant protein to mammalian cells, allowing expression of the recombinant protein, wherein the recombinant protein comprises at least one HLA-E ligand or a fragment, derivative or variant thereof capable of binding to HLA-E, isolating the expressed recombinant protein and formulating the isolated recombinant protein and a pharmaceutically acceptable carrier in a form suitable for administration.

In embodiments, in the method the recombinant protein can further contain HLA-E or a fragment, derivative or variant thereof.

In embodiments, in the method the recombinant protein can further contain a linker covalently associating the HLA-E ligand or the fragment, derivative or variant of HLA-E ligand capable of binding to HLA-E and the HLA-E or the fragment, derivative or variant of HLA-E.

In embodiments, in the method the HLA-E ligand or the fragment thereof can be identified via a method including contacting each of CMV-infected cell extract and CMV-uninfected cell extract with a plurality of HLA-E or a fragment, derivative or variant thereof that is immobilized on a substrate, allowing molecules in each of the cell extracts to bind to the plurality of the immobilized HLA-E or the fragment, derivative or variant thereof, collecting the molecules in each of the cell extracts that bind to the plurality of the immobilized HLA-E or the fragment, derivative or variant thereof, comparing the collected molecules from each of the cell extracts to identify molecules that are enriched in the CMV-infected cell extract as compared to the CMV-uninfected cell extract and determining the sequence of the enriched molecules.

In embodiments, in the method the HLA-E ligand or the fragment thereof can be identified via a method including contacting each of CMV-infected cell extract and CMV-uninfected cell extract with a plurality of HLA-E or a fragment, derivative or variant thereof that is immobilized on a substrate, allowing molecules in each of the cell extracts to bind to the plurality of the immobilized HLA-E or the fragment, derivative or variant thereof, collecting the molecules in each of the cell extracts that bind to the plurality of the immobilized HLA-E or the fragment, derivative or variant thereof, comparing the collected molecules from each of the cell extracts to identify molecules that are enriched in the CMV-infected cell extract as compared to the CMV-uninfected cell extract and determining the sequence of the enriched molecules.

In still another aspect, the present disclosure provides a method of inducing an adaptive immune response in a subject in need thereof. The method may include administering, to the subject, antibodies specific to HLA-E or a fragment, derivative or variant of HLA-E and/or a HLA-E ligand or a fragment, derivative or variant of HLA-E ligand that is capable of binding to HLA-E.

### BRIEF DESCRIPTION OF THE DRAWINGS

An understanding of certain features and advantages of the present disclosure will be obtained by reference to the following detailed description that sets forth illustrative embodiments, in which the principles of the disclosure are utilized, and the accompanying drawings of which:

FIG. 1 shows the results demonstrating expansion of NKG2C⁺ NK cells during acute CMV infection in hematopoietic stem cell transplant recipients. The data show percentage of NKG2C⁺ NK cells and mean fluorescence intensity (MFI) of NKG2C expression post-transplantation in recipients who reactivated CMV (open triangles) compared with control subjects who did not reactivate CMV (closed and open squares).

FIG. 2 shows the results demonstrating elevated frequency of the CD94-NKG2C⁺ CD57⁺ subset of NK cells in CMV-positive healthy adults and higher amounts (geometric Mean Fluorescence Intensity) of NKG2C on CD94-NKG2C⁺ NK cells in CMV-seropositive healthy adults. *, ** p<0.01.

FIG. 3 shows the results demonstrating secretion of sHLA-E*01:03 by CMV-infected cells. U-373MG cells stably transfected with sHLA-3*01:03 were infected with AD169 strain CMV and sHLA-E in the supernatant was measured by ELISA using anti-HLA-E mAb.

### DETAILED DESCRIPTION

The disclosure provides, inter cilia, compositions that contain ligands for HLA-E and/or CD94-NKG2C receptor that have enhanced immune properties. These ligands for HLA-E and/or CD94-NKG2C receptor can be used for cytomegalovirus (CMV) vaccine development. Methods of producing the composition and using the composition for enhancing immune response, increasing subsets of immune cells, modulating immune response are provided as well as uses for preventing, ameliorating and/or treating CMV infection.

### Definitions

Unless defined otherwise, all technical and scientific terms used herein have the same meaning as is commonly understood by one of skill in the art to which the claimed subject matter belongs. It is to be understood that the detailed descriptions are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

Reference in the specification to “some embodiments”, “an embodiment”, “one embodiment” or “other embodiments” means that a particular feature, structure, or characteristic described in connection with those embodiments is included in at least some embodiments, but not necessarily all embodiments, of the disclosure.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “polypeptide”, “peptide”, and “protein” are used interchangeably herein to designate a linear series of amino acid residues connected one to the other by peptide bonds, which series may include proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, (3-galactosidase, luciferase, and the like. Furthermore, it should be noted that a dash at the beginning or end of an amino acid sequence indicates either a peptide bond to a further sequence of one or more amino acid residues or a covalent bond to a carboxyl or hydroxyl end group. However, the absence of a dash should not be taken to mean that such peptide bond or covalent bond to a carboxyl or hydroxyl end group is not present, as it is conventional in representation of amino acid sequences to omit such.

The term “nucleic acid” is used herein in reference to either DNA or RNA, or molecules which contain deoxy- and/or ribonucleotides. Nucleic acids may be naturally occurring or synthetically made, and as such, include analogs of naturally occurring polynucleotides in which one or more nucleotides are modified over naturally occurring nucleotides.

The term “soluble” in the context of peptides or proteins may refer to peptides or proteins that do not form precipitants with their own peptide or protein sequences and/or other peptide or protein sequences in a solvent, e.g., an aqueous medium such as cytoplasm. In some instance, precipitants can involve aggregations.

The term “secretory” in the context of peptides or proteins may refer to a peptide or protein that tends to be exported outside a cell in which the peptide or protein was synthesized.

The terms “plasmid”, “vector”, “expression cassette” or “expression vector” refer to a nucleic acid molecule that encodes for one or more genes of interest and/or one or more regulatory elements necessary for the expression of the genes of interest.

The terms “conjugated” and “joining” generally refer to a chemical linkage, either covalent or non-covalent that proximally associates one molecule with second molecule.

The term “isolated” is intended to mean that a compound is separated from all or some of the components that accompany it in nature. “Isolated” also refers to the state of a compound separated from all or some of the components that accompany it during manufacture (e.g., chemical synthesis, recombinant expression, culture medium, and the like).

The term “purified” is intended to mean that a compound of interest is isolated and further enriched.

The term “non-naturally occurring” in the context of peptides or nucleic acids refers to a molecule that is not identical to a naturally occurring form thereof. For example, a non-naturally occurring peptide refers to a peptide sequence that is different from the form of the corresponding peptide present in nature. Thus, such non-naturally occurring peptide may include a peptide sequence having deletion, insertion, mutation and/or modification relative to its naturally occurring peptide sequence. Non-naturally occurring peptides can be chemically synthetized, produced in vitro (e.g., via an expression system using cultured cells or microorganisms), and/or isolated from nature and modified.

The term “recombinant” or “engineered” when used with reference, for example, to a cell, a nucleic acid, a protein, or a vector, indicates that the cell, nucleic acid, protein or vector has been modified by or is the result of laboratory methods. Thus, for example, recombinant or engineered proteins include proteins produced by laboratory methods. Recombinant or engineered proteins can include amino acid residues not found within the native (non-recombinant) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence.

The term “concentration” used in the context of a molecule such as peptide fragment refers to an amount of molecule, e.g., the number of moles of the molecule, present in a given volume of solution.

The phrase “specifically (or selectively) binds”, “binds with (high) specificity” or “binds with (high) affinity”, when used with reference to binding or association between two entities (e.g., a protein or peptide and its binding partner, or a nucleic acid sequence and its binding partner), refers to a binding reaction that determines the presence of the protein or peptide, or nucleic acid sequence, often in a heterogeneous population of such and other biologics. Thus, for example, ligands can bind to a particular protein at least two times the background and more typically more than 10 to 100 times background. Also, for example, the specified binding molecules such as nucleic acid-binding proteins or other nucleic acid sequence(s) bind to a particular nucleic acid sequence at a rate at least two tunes higher than the background and more typically more than 10 to 100 times higher than the background.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

The term “ligand” refers to an agent, e.g., a polypeptide or other molecule, capable of binding to its cognate binding molecule, or a complex thereof.

The terms “derivative” and “variant” refer without limitation to any compound or antibody that has a structure or sequence derived from the compounds and antibodies of the present disclosure and whose structure or sequence is sufficiently similar to those disclosed herein and that, based upon such similarity, would be expected by one skilled in the art to exhibit the same or similar activities and utilities as the claimed and/or referenced compounds or antibodies, thereby also interchangeably referred to “functionally equivalent” or as “functional equivalents”. Modifications to obtain “derivatives” or “variants” may include, for example, addition, deletion and/or substitution of one or more of the amino acid residues. The functional equivalent or fragment of the functional equivalent may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/Selenium-containing | Ser, Cys, Thr, Met |
| Cyclic | Pro |
| Aromatic | Phc, Tyr, Trp |
| Basic | His, Lys, Arg |
| Acidic and their Amide | Asp, Glu, Asn, Gln |

Conservative substitutions may be introduced in any position of a preferred predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. In some cases the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The term “treatment” used referring to a disease or condition is used herein to mean that at least an amelioration of the symptoms associated with the condition afflicting an individual is achieved, where amelioration is used in a broad sense to refer to at least a reduction in the magnitude of a parameter, e.g., a symptom, associated with the condition (e.g., CMV infection) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

The terms “effective amount”, “pharmaceutically effective amount”, or “therapeutically effective amount” as used herein mean a sufficient amount of the composition to provide the desired utility when administered to a subject having a particular condition. For instance, to elicit a desired response in a subject or individual such as preventing CMV infection and/or treating CMV infection and any associated symptoms, or eliciting an immune response, an effective amount of a vaccine composition is the amount that results in a substantial change in the occurrence (e.g., the rate and/or frequency) of CMV infection and/or the severity or length of CMV infection, when compared to an unvaccinated population or other negative control. The measurement of changes in in the occurrence of CMV infection and/or severity or length of CMV infection can be done by a variety of methods known in the art. In another example, for eliciting a favorable response in a subject to treat and/or prevent CMV infection, the effective amount is the amount which reduces, eliminates, or diminishes the symptoms associated with CMV infection. As will be understood by a person having ordinary skill in the art, the exact amount required will vary from subject to subject, depending on the species, age, and general condition of the subject, the severity of the condition or disease that is being treated, the particular composition used, its mode of administration, and the like. An appropriate effective amount can be determined by one of ordinary skill in the art using only routine experimentation.

The term “pharmaceutically acceptable excipient” as used herein refers to any suitable substance that provides a pharmaceutically acceptable carrier, additive or diluent for administration of a compound(s) of interest to a subject. “Pharmaceutically acceptable excipient” can encompass substances referred to as pharmaceutically acceptable diluents, pharmaceutically acceptable additives, and pharmaceutically acceptable carriers.

The terms “individual”, “subject”, or “host” as used herein refers to humans, mammals and other animals in the context of a CMV vaccine in the present disclosure. In some cases, the subject being a human can be a patient.

The term “immune response” refers to an immune response, e.g., the response of a cell of the immune system, such as a B cell, CD4+ T cell, CD8+ T cell, macrophage, natural killer (NK) cell, or monocyte, specific for a particular antigen (that is, an “antigen-specific response”). An immune response encompasses, for example, an increase of NK cell-mediated killing, proliferation of NK cells, an increase in the number of CD4+ or CD8+ T cells, increased killing of CMV-infected cells, T cell-mediated killing of infected cells and increased production or secretion of one or more cytokines. In the context of CMV infection, an immune response also encompasses increased proliferation of NK cells or T cells that express CD94-NKG2C receptor. The term “immune response” also encompasses a “protective immune response”, which inhibits a detrimental function or activity of a pathogen (such as CMV), reduces infection by the pathogen, or decreases symptoms (including death) that result from infection by the pathogen. A protective immune response can be measured, for example, by the inhibition of viral replication or plaque formation in a plaque reduction assay or ELISA neutralization assay (NELISA), or by measuring resistance to viral challenge in vivo in a standard experimental system. The term “immune response” also includes an “adaptive immune response”, also known as an “acquired immune response” in which adaptive immunity elicits immunological memory after an initial response to a specific pathogen, and leads to an enhanced response to that pathogen on subsequent encounters. The induction of immunological memory provides the basis of vaccination.

The term “immunogenic composition” refers to a composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic composition is a composition capable of eliciting an immune response in an immunocompetent subject. For example, an immunogenic composition can include one or more immunogenic epitopes of a CMV antigenic polypeptide. In addition, an immunogenic composition can include one or more of peptides of a host (or subject) that are specific to a CMV infection. Examples of such peptides from a host (or subject) specific for an CMV infection may include peptides having an increased expression level when the host is infected by CMV as compared to non-infection, and peptides that can bind to one or more of CMV antigenic polypeptides with specificity. In addition, an immunogenic composition can include isolated nucleic acid constructs (such as plasmids or viral vectors) that encode one or more immunogenic epitopes of an CMV antigenic polypeptide that can be used to express the epitope(s) (and thus be used to elicit an immune response against this polypeptide or a related polypeptide expressed by the pathogen) and/or the peptides of a host (or subject) that are specific to a CMV infection. In some embodiments, the immunogenic compositions comprise nucleic acid constructs encoding antigenic peptides and antigenic peptides in combination.

The terms “natural killer cell” or “NK cells” refer to a type of cytotoxic lymphocyte important to the innate immune system. NK cells provide generally rapid responses to viral-infected cells, and respond to tumor formation. Typically, some immune cells detect major histocompatibility complex (MHC) presented on infected cell surfaces, triggering cytokine release, causing lysis or apoptosis. NK cells have the ability to recognize stressed cells in the absence of antibodies and MHC, allowing for a faster immune reaction, but can also respond to infected or stressed cells coated with antibodies by a process named antibody-dependent cellular cytotoxicity (ADCC). NK cell receptor types (with inhibitory, as well as some activating members) can be differentiated by structure, including, but not limited to, the following: (1) activating receptors including some Ly49 receptors in rodents (homodimers) some killer-cell immunoglobulin-like receptors (KIRs) in humans, NCR (natural cytotoxicity receptors), CD94:NKG2C (heterodimers), and CD16 (FcγIIIA), and (2) inhibitory receptors including some KIRs in humans, some Ly49 receptors (homodimers) in rodents, and leukocyte immunoglobulin-like receptors (LIRs) in humans.

The terms “T lymphocyte” or “T cell” refer to a type of lymphocyte that plays a central role in cell-mediated immunity. T cells can be distinguished from other lymphocytes, such as B cells and NK cells, by the presence of a T-cell receptor on the cell surface. The several subsets of T cells each have a distinct function. The types of T cell include, but not limited to, effector T cells, helper T cells, cytotoxic killer T cells, memory T cells, regulatory T cells, natural killer T cell, mucosal-associated invariant T cells, alpha beta T cells, and gamma delta T cells.

The terms “B lymphocyte” or “B cell” refer to a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. B cells express B cell receptors (BCRs) (surface immunoglobulin) on their cell membrane. BCRs allow the B cell to bind a specific antigen, against which the B cell can then initiate an antibody response.

The terms “major histocompatibility complex” or “MHC” refer to a set of cell surface proteins involved in the acquired immune system to recognize foreign molecules in vertebrates, which in turn determines histocompatibility, immune tolerance, and initiates the adaptive immune response. The function of MHC molecules includes binding to antigens derived from pathogens and displaying them on the cell surface for recognition by the appropriate T-cells or natural killer cells. MHC molecules mediate interactions of leukocytes, also called white blood cells (WBCs), which are immune cells, with other leukocytes or with body cells. The human MHC is also called the HLA (human leukocyte antigen) complex (often just the HLA). The MHC gene family is divided into three subgroups: class I, class II, and class III.

The term “vaccine” refers to a biological composition that provides active acquired immunity to a particular disease or a pathogen. A vaccine typically contains one or more agents that induce an immune response in a host similar or identical to the immune response induced by a pathogen. Such agents are administered in a variety of forms, including, for example, as pathogen proteins, protein fragments, or peptides, as heat- or chemically-inactivated preparations of whole pathogen, as live attenuated preparations of whole pathogen, and the like. The agent stimulates the body's immune system to recognize the agent as a threat or indication of an infection, thereby inducing immunological memory so that the immune system can more easily recognize and destroy any of the pathogen on subsequent exposure. Vaccines can be prophylactic (example: to prevent or ameliorate the effects of a future infection by any natural or pathogen), or therapeutic (e.g., vaccines against cancer are being investigated). The administration of vaccines is referred to vaccination.

### Ligands for HLA-E Protein

NK cells and T cells bearing the invariant activating CD94-NKG2C receptor have been found to specifically respond to CMV infection in the outbred human population. CD94-NKG2C recognizes HLA-E, a non-polymorphic MHC class I protein expressed on essentially all cells. The CD94-NKG2C recognition of HLA-E/peptide complexes on healthy, uninfected cells is insufficient to activate and expand NK cells or T cells. Alternations in the HLA-E protein repertoire in CMV-infected cells may result in high and/or specific binding to CD94-NKG2C that can induce NK cell activation and/or proliferation. Accordingly, in one aspect, the invention is directed to the identification of physiological ligands of the activating CD94-NKG2C receptor that can allow NK cells to specifically respond to CMV infection and use of the ligands for a novel and/or effective CMV vaccine.

HLA-E, the major histocompatibility complex class I protein, is highly invariant in the human population. Like other HLA proteins, HLA-E is a heterodimer composed of an alpha heavy chain and a light chain (beta-2-microglobulin), and peptides bound to the peptide-binding groove. The activating CD94-NKG2C receptor present on NK cells recognizes and binds to HLA-E with specificity. Therefore, in one aspect, this disclosure relates to the identification of HLA-E ligands that bind either with high affinity or preferentially to the activating CD94-NKG2C receptor, which can activate the NK cell response for control of CMV infection. Identification of a ligand for HLA-E protein and/or CD94-NKG2C receptor can provide a unique opportunity to incorporate this ligand into CMV vaccines to augment the NK cell response, complementing the immunity provided by B and T cells. CD94-NKG2C receptors are expressed by a subset of CD8+ T cells, in addition to NK cells; therefore, vaccination with a CD94-NKG2C ligand can also enhance protective T cell responses.

In certain embodiments, a ligand for HLA-E protein can be identified and used as a CMV vaccine. In certain embodiments, the ligand may show increased binding to HLA-E protein in CMV-infected cells as compared to uninfected cells. Such increase in binding can be due to increased expression of the ligand and/or increased affinity to HLA-E protein specific to CMV-infection. Therefore, in some embodiments, such ligands can be identified via comparison of the peptide repertoire in the CMV-infected cells and uninfected cells and identification of those showing increased binding to HLA-E protein from CMV-infected cells.

In some embodiments, the ligands for HLA-E protein can include peptides derived from a host or subject. For example, there can be host-derived peptides that are either more highly expressed or uniquely present in CMV-infected cells due to the cellular stress caused by infection. Such ligands may specifically bind to HLA-E protein in cells infected with CMV, inducing an immune response to infection.

In some embodiments, the ligands for HLA-E protein may include any biological components of CMV, i.e., CMV-derived components. In certain embodiments, the viral components that can specifically bind to HLA-E protein include viral peptides.

In some embodiments, the ligands for HLA-E protein can include more than one binding partner such that different complexes comprising HLA-E protein and its binding partners (or ligands) may comprise two or more different types of peptides. For example, HLA-E protein can form a complex with one or more ligand(s) that is(are) derived from a host and one or more ligand(s) that is(are) derived from CMV. Alternatively, HLA-E protein can form a complex with two or more of host-derived peptides, or with two or more of peptides derived from CMV. In some embodiments, each of HLA-E protein and its ligands according to the invention can be of a naturally occurring or non-naturally occurring form. In some embodiments, any of the peptides used in the CMV vaccine compositions provided herein including HLA-E protein and its ligands can be synthesized using a technique or method known in the art.

### Method of Identification of HLA-E Ligands

Ligands for HLA-E protein can be identified. In some embodiments, immunoproteomics techniques can be used to identify ligand/HLA-E protein complex(es) that are either unique to or enriched in CMV-infected cells as compared to uninfected cells. In some embodiments, an HLA-E protein used to identify its ligand(s) can be in a naturally occurring or non-naturally occurring form. In some embodiments, secreted and/or soluble forms of HLA-E proteins (e.g. sHLA-E) can be expressed in vitro. Cultures expressing secreted and/or soluble forms of HLA-E can be infected with CMV or remain uninfected. sHLA-E peptides from CMV-infected cells and uninfected cells can be collected and passed over columns coupled to an antibody capable of capturing a complex containing HLA-E proteins. Peptide-containing fractions from each group of cells, i.e., CMV-infected cells and uninfected cells, are eluted, separated, and comparatively analyzed, e.g., by mass spectrometry. The peptide repertoires identified from each group of cells can be comparatively analyzed and candidate ligands can be sequenced for identification. Comparison of the peptide repertoire in the infected and uninfected cells can identify not only viral peptides but also host-derived peptides in infected cells, which may be detected due to the cellular stress resulting from viral infection. See, e.g. Table 1.

In some embodiments, CMV-infection can induce formation of HLA-E/host-encoded peptide or HLA-E/CMV-encoded peptide complexes generating high affinity ligands for (1) the CD94-NKG2C receptor that is present on NK cells or T cells and/or (2) T cell antigen receptors. To identify CMV-induced HLA-E/peptide complexes that preferentially bind to the activating CD94-NKG2C receptor or T cell receptors, novel host-derived or CMV-derived peptides bound to HLA-E can be identified experimentally in CMV-infected cells. HLA-E ligands unique to CMV-infected cells and host-derived peptides over-represented in CMV-infected cells can be selected by comparing the repertoire of peptides from uninfected and CMV-infected cells for their ability to affect the binding of HLA-E to either CD94-NKG2A or CD94-NKG2C. In one embodiment, among the tested candidates, peptides that demonstrate specific or preferential binding to CD94-NKG2C can be used for producing CMV vaccine compositions. In some other embodiments where the T cells specific immune responses are concerned, the repertoire of peptides can be compared from uninfected versus infected cells peptides that are unique to CMV-infected cells and host-derived peptides that are over-represented in infected cells for their ability to affect the binding of HLA-E to one or more of T cell antigen receptors can be selected. Some examples of T cell antigen receptors that can be tested herein include, but not limited to, receptors for antigen CD3, CD8, TCRαβ, CD25, LAG3, CD39, CTLA4, CD45RA, CD45RO, CCR7, CD27, CD28, CD56, VD57, CD62L or CD94. Further examples of T cell antigen receptors that can be used in various embodiments of the present disclosures can be found, for example, from Joosten et al., “Characteristics of HLA-E Restricted T-Cell Responses and Their Role in Infectious Diseases”, Journal of Immunology Research, Volume 2016, Article ID 2695396, 11 pages, the content of which is incorporated by reference in the present application. In addition, in another embodiment, HLA-E proteins and its ligand(s) can be tested for the ability to activate and/or induce proliferation of NK cells expressing CD94-NKG2C, the ability to activate and/or induce proliferation of T cells, or the ability to induce secretion of antibodies against the CMV peptides by B cells by vaccination with CMV peptides bound to HLA-E. In some embodiments, peptides that show specific or preferential binding to CD94-NKG2C and/or are capable of activating NK cells expressing CD94-NKG2C or activating T cells can be further chosen as preferred candidates for CMV vaccine compositions and methods of using the same.

In some embodiments, TAP-deficient mouse RMA-S cells transfected with human HLA class I molecules can be used to determine binding of peptides. HLA-E/peptide complexes to be screened for CD94-NKG2A and CD94-NKG2C binding can be initially selected by a comparison of the peptides eluted from HLA-E derived from CMV-infected versus uninfected cells. CMV-induced host-derived peptides, as well as viral peptides, are of interest and can be evaluated. All viral peptides identified by HLA-E binding can be analyzed. The identified peptides can include: 1) Peptides that bind both CD94-NKG2A and CD94-NKG2C, but with higher affinity for CD94-NKG2A, such as the leader peptide from the CMV UL40 protein, 2) Peptides that bind to the inhibitory CD94-NKG2A, but not the CD94-NKG2C receptor, 3) Peptides that bind neither CD94-NKG2A nor -NKG2C, such as the hsp60 peptide, and 4) Of interest may be peptides that bind to CD94-NKG2C with high affinity (even if they are also able to bind CD94-NKG2A) or possibly peptides that bind to CD94-NKG2C and not CD94-NKG2A. Peptides that bind to CD94-NKG2C with high affinity may activate and clonally expand NK cells, even if they also bind to CD94-NKG2A with high affinity because this would allow the preferential expansion of CD94-NKG2C+, NKG2A-negative (“single-positive”) NK cells. Peptides that demonstrate specific or preferential binding to CD94-NKG2C can be used to produce HLA-E-peptide tetramers for validation and further analysis at least in some embodiments. The NIH Tetramer Core Facility at Emory University can produce custom tetramers of HLA-E*01:01 (GenBank: AAA59835.1; SEQ ID NO: 1) and HLA-E*01:03 (NCBI Reference Sequence: NP_005507.3; SEQ ID NO: 2) (http://tetramer.yerkes.emory.edu/reagents/request-process).

The fluorochrome-labeled HLA-E-peptide tetramers can be used to stain human NK cells expressing the CD94-NKG2A and CD94-NKG2C receptors or T cells bearing T cell receptors that bind to the HLA-E-peptide tetramers to confirm these as functional ligands.

### Compositions for Modulating Immune Responses

In one aspect, provided herein are HLA-E ligands useful in the preparation of vaccine compositions. In some embodiments, the HLA-E ligands can also bind to CD94-NKG2C receptor with high affinity, thereby forming a complex having HLA-E ligand, HLA-E protein and CD94-NK2GC receptor. In some embodiments, the vaccine compositions comprise HLA-E ligands suitable for use in a CMV vaccine. The vaccine composition may further comprise an agent that can induce an immune response in a host after vaccination that is similar to or mimics a CMV-specific immune response sufficient to permit the host to acquire immunity to CMV. In some embodiments, the agent may contain HLA-E protein or any fragment thereof.

In some embodiments, a vaccine composition can have one or more HLA-E ligands or any fragments, derivatives or variants thereof. In some embodiments, the vaccine composition can have one or more full-length HLA-E ligand or any fragment, derivative or variant thereof that retains the ability to bind HLA-E protein. In some embodiments, an HLA-E ligand contained in a CMV vaccine composition can be different from its naturally occurring, full-length form.

The HLA-E ligands or any fragments, derivatives or variants thereof for CMV vaccine may be capable of binding to HLA-E protein and/or activating receptor of CD94-NKG2C or T cell receptor on NK cells or T cells and inducing an immune response. The binding affinity of the HLA-E ligands or any fragments, derivatives or variants thereof to HLA-E protein and/or the activating CD94-NKG2C receptor can vary in that there can be about 10%, 20%, 30%, 40%, 50%, 60%, 70%, 80%, 90%, 95% or 99% increase or decrease in the binding affinity as compared to that of the full-length HLA-E ligands. In addition, the activation of the NK cell or T cell immune response caused by the vaccine composition having the HLA-E ligands or any fragments, derivatives or variants thereof can also vary in that there can be about 10%, 20%, 30%, 40%, 50%, 60%, 70%, 80%, 90%, 95% or 99% increase or decrease in the immune response activity as compared to that of a full-length HLA-E ligands.

The HLA-E ligands or any fragments, derivatives or variants thereof for CMV vaccine may be associated with HLA-E protein (or any fragments, derivatives or variants thereof) when binding to a) a CD94-NKG2C receptor that is present on NK cells or T cells; or b) T cell antigen receptors.

In some embodiments, the HLA-E ligands or any fragments, derivatives or variants thereof contained in a CMV vaccine do not substantially bind to an inhibitory CD94-NKG2A receptor of NK cells or T cells. In some embodiments, the binding affinity of the HLA-E protein or any fragments, derivatives or variants thereof to the CD94-NKG2A receptor, as compared to that to the activating CD94-NKG2C receptor, can be about at least 10%, 20%, 30%, 40%, 50%, 60%, 70%, 80%, 90%, 95% or 99% decreased.

In some embodiments, the HLA-E ligands or any fragments, derivatives or variants for CMV vaccine can have at least 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, 95 amino acids, 100 amino acids, 105 amino acids, 110 amino acids, 115 amino acids, 120 amino acids, 125 amino acids, 130 amino acids, 135 amino acids, 140 amino acids, 145 amino acids, 150 amino acids, 155 amino acids, 160 amino acids, 165 amino acids, 170 amino acids, 175 amino acids, 180 amino acids, 185 amino acids, 190 amino acids, 195 amino acids, 200 amino acids, 205 amino acids, 210 amino acids, 215 amino acids, 220 amino acids, 225 amino acids, 230 amino acids, 235 amino acids, 240 amino acids, 245 amino acids, 250 amino acids, 255 amino acids, 260 amino acids, 265 amino acids, 270 amino acids, 275 amino acids, 280 amino acids, 285 amino acids, 290 amino acids, 295 amino acids, 300 amino acids, more than 300 amino acids or any intervening number of amino acids thereof in length.

The ligands for HLA-E protein can be host-derived peptides or CMV-derived peptides. In some embodiments, the ligands of HLA-E protein contained in a CMV vaccine composition can be wild-type, full length peptides or any fragments, derivatives or variants thereof. In embodiments, the vaccine compositions can contain a plurality of component among those set forth above, therefore generating a broader response by T cells, B cells, and/or NK cells.

In some embodiments, a vaccine composition comprises one or more HLA-E ligands. In some embodiments, the vaccine composition comprises 1, 2, 3, 4, 5, 6, 7, 8, 9, 10, 11, 12, 13, 14, 15, 16, 17, 18, 19, 20, 25, 30, or more HLA-E ligands. In some embodiments, the HLA-E ligands comprise one or more host-derived peptides. In some embodiments, the HLA-E ligands comprise one or more CMV-derived peptides.

In some embodiments, a vaccine composition has a fragment, derivative or variant of HLA-E ligands. Such fragment, derivative or variant can have a peptide sequence having at least 50%, 60%, 70%, 80%, 90%, 91%, 92%, 93%, 94%, 95%, 96%, 97%, 98%, 99%, or 100% sequence identity to the full length of the ligand peptides.

In some embodiments, a vaccine composition can have one or more HLA-E proteins or any fragments thereof. In some embodiments, the vaccine composition can have one or more full-length HLA-E protein or any fragment, derivative or variant thereof that retains the ability to bind its ligands. In some embodiments, an HLA-E protein contained in a CMV vaccine composition can be a soluble and/or secretory form of an HLA-E protein, which can be different from its naturally occurring, full-length form. For example, in some embodiments, a truncated soluble HLA-E*01:03 construct (SEQ ID NO:2) can be transfected into U373 cells with and the cells are infected with CMV. Then the HLA-E/ligand complexes can be isolated from the supernatant and the ligand (e.g. peptides binding to HLA-E) are eluted and sequenced them by mass spectrometry. The sequence of the soluble truncated HLA-E protein expressed in U373, sHLA-E*0103 tVLDLr, is provided in SEQ ID NO: 3.

The fragment, derivative or variant of HLA-E protein for CMV vaccine may be capable of binding to its ligand(s) and/or activating receptor of CD94-NKG2C or T cell receptor on NK cells or T cells and inducing an immune response. The binding affinity of the fragment, derivative or variant of HLA-E protein to its ligand(s) and/or the activating CD94-NKG2C receptor can vary in that there can be about 10%, 20%, 30%, 40%, 50%, 60%, 70%, 80%, 90%, 95% or 99% increase or decrease in the binding affinity as compared to that of the full-length HLA-E protein. In addition, the activation of the NK cell or T cell immune response caused by the vaccine composition having the fragment, derivative or variant of HLA-E protein can also vary in that there can be about 10%, 20%, 30%, 40%, 50%, 60%, 70%, 80%, 90%, 95% or 99% increase or decrease in the immune response activity as compared to that of a full-length HLA-E protein.

In some embodiments, the fragment, derivative or variant of HLA-E protein for CMV vaccine can have at least 5 amino acids, 10 amino acids, 15 amino acids, 20 amino acids, 25 amino acids, 30 amino acids, 35 amino acids, 40 amino acids, 45 amino acids, 50 amino acids, 55 amino acids, 60 amino acids, 65 amino acids, 70 amino acids, 75 amino acids, 80 amino acids, 85 amino acids, 90 amino acids, 95 amino acids, 100 amino acids, 105 amino acids, 110 amino acids, 115 amino acids, 120 amino acids, 125 amino acids, 130 amino acids, 135 amino acids, 140 amino acids, 145 amino acids, 150 amino acids, 155 amino acids, 160 amino acids, 165 amino acids, 170 amino acids, 175 amino acids, 180 amino acids, 185 amino acids, 190 amino acids, 195 amino acids, 200 amino acids, 205 amino acids, 210 amino acids, 215 amino acids, 220 amino acids, 225 amino acids, 230 amino acids, 235 amino acids, 240 amino acids, 245 amino acids, 250 amino acids, 255 amino acids, 260 amino acids, 265 amino acids, 270 amino acids, 275 amino acids, 280 amino acids, 285 amino acids, 290 amino acids, 295 amino acids, 300 amino acids, more than 300 amino acids or any intervening number of amino acids thereof in length.

In some embodiments, a vaccine composition can have a fragment, derivative or variant of HLA-E protein. Such fragment, derivative or variant can have a peptide sequence having at least 50%, 60%, 70%, 80%, 90%, 91%, 92%, 93%, 94%, 95%, 96%, 97%, 98%, 99%, or 100% sequence identity to HLA-E protein presented in SEQ ID NO. 1, 2 or 3.

HLA-E protein or any fragments, derivatives or variants thereof may be associated its ligand(s) (or any fragments, derivatives or variants thereof) when binding to a) a CD94-NKG2C receptor that is present on NK cells or T cells; or b) T cell antigen receptors.

In some embodiments, a vaccine composition can have a recombinant peptide in which a HLA-E protein can be fused to one or more of its ligand(s). The HLA-E protein and the ligands thereof present in the recombinant peptide can be full length peptides or any fragments, derivatives or variants thereof, respectively. Each of recombinant peptide can contain more than one molecule of each of HLA-E peptide and/or its ligand(s). Thus, for example, the copy number ratio between HLA-E peptide (or any fragment, derivative or variant thereof) and its ligand(s) (or any fragment, derivative or variant thereof) present in a single recombinant peptide can be about 1:1, 1:2, 1:3, 1:4, 1:5, 1:10, 1:20, 1:30, 1:50 or more and any intervening range of the foregoing, or vice versa. In some embodiments, any immunogenic agents for CMV, e.g. HLA-E protein (or any fragment, derivative or variant thereof) and its ligand(s) (or any fragment, derivative or variant thereof), present in a recombinant peptide can be linked via a linker. Examples of such linker may include, but not limited to, a linker of 15 residues (G₄S)₃, a linker of 20 residues (G₄S)₄ and a linker of 25 residues (G₄S)₅. Any other linker sequences known in the art, see, e.g. Yu et al., “Cutting Edge: Single-Chain Trimers of MHC Class I Molecules Form Stable Structures That Potently Stimulate Antigen-Specific T Cells and B Cells”, J. Immunol. 2002; 168:3145-3149, Kotsiou et al., “Properties and Applications of Single-Chain Major Histocompatibility Complex Class I Molecules”, Antioxid Redox Signal. 2011 Aug. 1; 15(3): 645-655, Tafuro et al., “Reconstitution of antigen presentation in HLA class I-negative cancer cells with peptide-beta2m fusion molecules.” Eur J Immunol 31:440-449, 2001, and Kim et al. “Single-Chain HLA-A2 MHC Trimers That Incorporate an Immundominant Peptide Elicit Protective T Cell Immunity against Lethal West Nile Virus Infection”, J. Immunol. 2010, 184(8): 4423-4430, content of which are incorporated by reference in the present application, can also be used in generating recombinant peptides of the present disclosure. In some embodiments, a recombinant peptide may contain HLA-E protein, its ligand and human β2-microglobulin as it can provide a conformation that can bind to T cell receptor or the NK receptors.

In some embodiments, the vaccine composition is delivered as a nucleic acid, rather than as a peptide or mixture of peptides, e.g., as an expression vector that encodes one or more agents which induce an immune response specific to CMV, i.e., immunogenic agents for CMV. In some embodiments, the nucleic acid comprises genes encoding an HLA-E ligand (or any fragment, derivative or variant thereof) and/or HLA-E protein (or any fragment, derivative or variant thereof). Alternatively, the response is a B cell response, and results in the production of specific antibodies. In some embodiments, the vaccine composition comprises a nucleic acid encoding an HLA-E ligand (or any fragment, derivative or variant thereof) and one or more immunogenic agents in peptide form in combination.

In some embodiments, a vaccine composition according to the disclosure herewith can be formulated as a CMV vaccine. The vaccine composition can induce an immune response. In some embodiments, an immune response can include a T cell response, such as a CD4+ response or a CD8+ response. In some embodiments, an immune response can include increase of NK cell-mediated killing, proliferation of NK cells, increase of T cells, killing of CMV-infected cells, T cell-mediated killing of CMV-infected cells and/or increased production or secretion of one or more cytokines, e.g. interferon gamma or production of antibodies by B cells reactive with the CMV peptides. In some embodiments, the NK cells and/or T cells affected by the CMV vaccination express the CD94-NKG2C receptor. In some embodiments, the NK cells affected by the CMV vaccination can be specific for CMV-infected cells or in other embodiments the vaccine will engage the T cell receptor on T cells to initiate a T cell response. In some embodiments, the vaccine composition can also induce an antibody response by B cells that can recognize the HLA-E protein and its ligand complex on the surface of CMV-infected cells and allow destruction of these infected cells by complement fixation or induction of antibody-dependent cell-mediated cytotoxicity (ADCC) by NK cells or myeloid cells. Furthermore, in some embodiments, antibodies that specifically bind to the HLA-E and its ligand complex expressed on the surface of CMV infected cells can be used to generate a chimeric antigen receptor (CAR) for introduction into T cells or NK cells for adoptive cell therapy of CMV infection.

In some embodiments, the vaccine composition according to the disclosure can induce T cell specific responses. This induction can be in part via specific recognition of HLA-E protein by any receptors on T cells. Accordingly, the vaccine can generate T-cell mediated immunity, functioning as a T cell vaccine against CMV infection. The T cell response induced by the vaccine, e.g. HLA-E and its ligand complex can also help the generation of antibodies by B cells against these peptides that may in turn be neutralizing.

### Pharmaceutical Formulations

Compositions that include HLA-E ligands can be formulated for administration to human subjects infected with CMV. In one embodiment, compositions for CMV infection further contain a pharmaceutically acceptable excipient and/or a pharmaceutically acceptable carrier, forming a pharmaceutical composition or formulation. In another embodiment, compositions for treating and/or preventing CMV infection can be produced to be sterile compositions.

Pharmaceutical compositions of the present disclosure containing one or more immunogenic agents that induce a CMV-specific immune response, e.g., HLA-E ligands (or any fragment, derivative or variant thereof) and/or HLA-E proteins (or any fragment, derivative or variant thereof) as an active ingredient comprise pharmaceutically acceptable excipients or additives depending on the route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used can be chosen from, but not limited to, the above or combinations thereof, as appropriate, depending on the dosage form of the present disclosure.

Formulation of the pharmaceutical compositions of the present disclosure can vary according to the route of administration selected (e.g., solution, emulsion). Parenteral administration means any non-oral means of administration, and is generally interpreted by those skilled in the art as relating to direct injection into the body, bypassing the skin and mucous membranes. Common parenteral routes of administration are intramuscular (IM), subcutaneous (SC), and intravenous (IV). See, e.g., https://www.nursingtimes.nct/administration-of-drugs-3-parenteral/5034777.article. An appropriate composition comprising the active ingredient(s) to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, saline, glycine, and the like, may include proteins for enhanced stability, such as albumin, lipoprotein, globulin, etc. In some embodiments, those proteins are subjected to mild chemical modifications or the like.

Therapeutic formulations for CMV vaccines described herein can be prepared for storage by mixing the active ingredients, i.e., immunogenic agent(s) having the desired degree of purity with optional physiologically acceptable carriers, excipients or stabilizers. Acceptable carriers, excipients, or stabilizers can be nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The vaccine composition may also be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulation herein may also contain more than one active compound (e.g., a second active agent in addition to the immunogenic agent(s)), which may be selected to complementary activities that do not adversely affect each other. Such molecules can be suitably present in combination in amounts that can be effective for the purpose intended.

### Methods of Making Compositions for Modulating Immune Responses

In another aspect, provided herein are methods of making compositions for modulating immune responses in a subject. The HLA-E proteins and/or its ligands described herein can be used to make compositions for modulating immune responses. In one embodiment, the composition is a CMV vaccine which contains at least one non-naturally occurring HLA-E ligand or any fragment, derivative or variant thereof capable of binding to HLA-E. The method may comprise formulating the at least one non-naturally occurring HLA-E ligand or any fragment, derivative or variant thereof capable of binding to HLA-E and a pharmaceutically acceptable carrier in a form suitable for administration. In certain embodiments, the composition can be formulated to contain an HLA-E ligand or any fragment, derivative or variant thereof that is capable of binding to HLA-E protein in the vaccine composition.

In some embodiments, a method of making a composition to induce an immune response, e.g., a CMV vaccine composition, can utilize a nucleic acid structure such as an expression system. The method may comprise introducing a vector sequence encoding a recombinant protein to mammalian cells, thereby allowing expression of the recombinant protein. The recombinant protein can contain at least one non-naturally occurring HLA-E ligand or any fragment, derivative or variant thereof capable of binding to HLA-E protein (or any fragment, derivative or variant thereof). The expressed recombinant protein can then be isolated and formulated with a pharmaceutically acceptable carrier in a form suitable for administration. In some embodiments, the recombinant protein further contains HLA-E protein or any fragment, derivative or variant thereof that is capable of binding to its ligand (or any fragment, derivative or variant thereof). In certain embodiments, the recombinant protein can further contain a linker covalently associating the HLA-E ligand (or any fragment, derivative or variant thereof) and the HLA-E protein (or any fragment, derivative or variant thereof).

### Methods of Inducing an Immune Response

In another aspect, provided herein are methods of inducing an immune response in a subject. In some embodiments, the methods may comprise treating and/or preventing CMV using a CMV vaccine. The methods generally involve administering an individual in need thereof a therapeutically effective amount of a CMV vaccine composition or a pharmaceutical composition comprising the vaccine composition described herein, alone (e.g., in monotherapy) or in combination (e.g., in combination therapy) with one or more additional ingredients, e.g., a pharmaceutically acceptable excipient and/or additional therapeutic agent.

In some embodiments, desired activities of CMV vaccine compositions according to the present disclosure include treatment and/or prevention of CMV infection. Treatment may include an amelioration or reduction in the magnitude of a parameter of symptoms associated with CMV infection (e.g., severity or length of infection). The CMV vaccine composition can also prevent infection by, for example, reducing the risk of development of clinical symptoms, including causing the clinical symptoms not to develop, e.g., preventing disease progression and inhibition that is, arresting the development or further development of clinical symptoms. In some embodiments, the desired activities of the CMV vaccines may include one or more of induction of an immune response such as preferential expansion of NK cells, increase of T cells expressing CD94-NKG2C in a subject and decrease of viral load of CMV in a subject. In some embodiments, the cells expressing CD94-NKG2C cells, which can be increased in proliferation by CMV vaccination, include NK cells and/or T cells. In other embodiments, the CMV vaccine may interact with T cell receptors on T cells and stimulate T cells to proliferate, produce cytokines, and kill CMV-infected cells. Other embodiments may include the CMV vaccine inducing antibodies against the CMV peptide or the HLA-E/CMV peptide complex.

In some embodiments, the vaccination may also include induction of an adaptive immune response in a subject in need thereof by administering specific antibodies. The method may comprise administering antibodies against one or more of immunogenic agents, e.g., HLA-E ligands (or any fragments, derivatives or variants thereof) and HLA-E proteins (or any fragments, derivatives or variants thereof), individually or in combination, to a subject who may need to acquire an adaptive immune response. The antibodies can be used for future chimeric antigen receptors for introduction into T cells or NK cells for adaptive cell therapy using standard procedures to generate chimeric antigen receptors. In some embodiments, the antibodies for adaptive cell therapy can be produced by administering one or both of at least one non-naturally occurring HLA-E ligands (or any fragments, derivatives or variants thereof) and HLA-E proteins (or any fragments, derivatives or variants thereof) to a subject, who may or may not be the subject in need to adaptive cell therapy. In some embodiments, the subject used for antibody production can be a separate individual or non-human animal. Once the subject is administered with the immunogenic agents and antibodies specific for the agents can be produced in the subject, the antibodies can be isolated from the subject. Antibodies can be generated against the HLA-E/CMV peptide complex by using standard techniques (immunization of mice or rabbits, immunization of transgenic mice having human immunoglobulin genes, antibody phage display techniques, and other well established procedures). The isolated antibodies can be processed suitable for administration to a subject who is in need of adaptive cell therapy. Alternatively, the immunogenic agents can be administered to cultured cells so that the antibodies specific for the immunogenic agents can be produced in vitro. The antibodies from the cultured cells can be isolated and processed for administration to a subject in need of adaptive cell therapy.

Antibodies can be prepared using a wide variety of techniques known in the art including the use of hybridoma, recombinant, and phage display technologies, or a combination thereof. For example, antibody may be made and isolated using methods of phage display. The antibody may also be isolated from sera of an animal host immunized with an immunogenic composition comprising an antigen (e.g. an effector antigen or a guide antigen), which encompasses whole proteins and fragments thereof. Exemplary antibodies include an isolated antibody capable of specifically binding to the antigen or fragments thereof.

The antigen that coats the wells for phage display panning or the immunogenic composition used to elicit the antibody of the present disclosure may comprise an aggregate of one or more antigens. The method may involve exposing antigens to an aggregating condition so as to form an aggregate. Thus the methods of production described above may further include a step of forming an aggregate of the isolated antigens. Examples of the aggregating conditions include heating, addition of an excipient that facilitates aggregation, and the like.

Antigens used to coat the wells for phage panning or to elicit antibodies of the present disclosure may be conjugated to another molecule. For example, the antigen can be conjugated to a second molecule such as a peptide, polypeptide, lipid, carbohydrate and the like that aids in solubility, storage or other handling properties, cell permeability, half-life, controls release and/or distribution such as by targeting a particular cell (e.g., neurons, leucocytes etc.) or cellular location (e.g., lysosome, endosome, mitochondria etc.), tissue or other bodily location (e.g., blood, neural tissue, particular organs etc.).

Antibodies, including antigen binding fragments of antibodies, may also be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library can be constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Any of the antibodies described herein may also be in the form of an antibody fragment.

Antibody fragments comprise a portion of an intact full length antibody and can include an antigen binding or variable region of the intact antibody. Examples of antibody fragments include Fab; Fab′; F(ab′)2; Fv fragments; diabodies; linear antibodies; single-chain antibody molecules (e.g., scFv); multispecific antibody fragments such as bispecific, trispecific, etc. antibodies (e.g., diabodies, triabodies, tetrabodies); minibody; chelating recombinant antibody; tribodies or bibodies; intrabodies; nanobodies; small modular immunopharmaceuticals (SMIP), binding-domain immunoglobulin fusion proteins; camelized antibodies; VHH containing antibodies; and other polypeptides formed from antibody fragments.

Methods of making antibody fragments are known in the art (see for example, Harlow and Lane, Antibodies: A Laboratory Manual, Cold Spring Harbor Laboratory, N Y, 1988, incorporated herein by reference). Antibody fragments can be prepared by proteolytic hydrolysis of the antibody or by expression in cells encoding the fragment. Antibody fragments can be obtained by, e.g. pepsin or papain digestion of whole antibodies conventional methods. For example, antibody fragments can be produced by enzymatic cleavage of antibodies with pepsin to provide a 5 S fragment denoted F(ab′)2. This fragment can be further cleaved using a thiol reducing agent, and optionally a blocking group for the sulfhydryl groups resulting from cleavage of disulfide linkages, to produce 3.5 S Fab′ monovalent fragments. Alternatively, an enzymatic cleavage using pepsin produces two monovalent Fab′ fragments and an Fc fragment directly.

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The disclosures contemplate human and humanized forms of non-human (e.g. murine) antibodies. Such humanized antibodies are chimeric immunoglobulins, immunoglobulin chains or fragments thereof (such as Fv, Fab, Fab′, F(ab′)2 or other antigen-binding subsequences of antibodies) that contain a minimal sequence derived from non-human immunoglobulin, such as the epitope recognizing sequence. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity. Humanized antibody(es) containing a minimal sequence(s) of antibody(es) of the invention, such as a sequence(s) recognizing the epitope(s) described herein, is a preferred embodiment of the invention.

In embodiments, a subject or individual in need of CMV vaccination has or is suspected of being infected with CMV. In some other embodiments, a subject or individual in need of CMV vaccination is not infected with CMV, but may be at risk of exposure to CMV and the vaccination can reduce likelihood of CMV infection in the subject. In some embodiments, a subject or individual in need of CMV vaccination does not have CMV infection. In some embodiments, the subject can be a child or an infant. In some embodiments, the subject can be women prior to pregnancy. In some other embodiments, the subject has a compromised immune system or can be a transplant patient.

In some embodiments, an effective amount of CMV vaccines or pharmaceutical formulations thereof according to the present disclosure can be administered to an individual in need thereof. The amount administered varies depending upon the goal of the administration, the health and physical condition of the individual to be treated, age, the taxonomic group of individual to be treated (e.g., human, non-human primate, primate, etc.), the degree of resolution desired, the formulation of the vaccine composition or pharmaceutical formulation thereof, the treating clinician's assessment of the medical situation, and other relevant factors. It is expected that the amount can fall in a relatively broad range that can be determined through routine trials. For example, the amount of the vaccine compositions or pharmaceutical formulations thereof employed to treat and/or prevent CMV infection cannot be more than about the amount that could otherwise be irreversibly toxic to the subject (i.e., maximum tolerated dose). In other cases the amount can be around or even well below the toxic threshold, but still in an immuno-effective concentration range, or even as low as threshold dose.

Individual doses can be typically not less than an amount required to produce a measurable effect on the individual, and may be determined based on the pharmacokinetics and pharmacology for absorption, distribution, metabolism, and excretion (“ADME”) of the vaccine compositions or pharmaceutical formulations thereof, and thus based on the disposition of the composition within the individual. This includes consideration of the route of administration as well as dosage amount, which can be adjusted for, e.g., parenteral (applied by routes other than the digestive tract for systemic or local effects) applications. For instance, administration of the CMV vaccines or pharmaceutical formulations thereof can be via injection and often intravenous, intramuscular, or a combination thereof.

The vaccine composition or pharmaceutical formulation thereof may be administered by infusion or by local injection, e.g., by infusion at a rate of about 10 mg/h to about 200 mg/h, about mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m²/day to about 10 mg/m²/day, including about 1 mg/m²/day to about 9 mg/m²/day, about 2 mg/m²/day to about 8 mg/m²/day, about 3 mg/m²/day to about 7 mg/m²/day, about 4 mg/m²/day to about 6 mg/m²/day, about 4.5 mg/m²/day to about 5.5 mg/m²/day. Administration can be repeated over a desired period, e.g., repeated over a period of about 1 day to about 5 days or once every several days, for example, about five days, over about 1 month, about 2 months, etc. The weight herein can be a weight of immunogenic agents (e.g., HLA-E ligands (or any fragments, derivatives or variant thereof) and/or HLA-E proteins (or any fragments, derivatives or variant thereof) or a weight of a CMV vaccine composition or pharmaceutical formulation thereof

In some embodiments, CMV vaccine compositions or pharmaceutical formulations thereof can be administered to a subject in need thereof before the subject is exposed to or infected with CMV. For example, the subject can be a young child or infant who was not previously infected with CMV. In such a case, the child or infant can be vaccinated, e.g., immediately after birth or with first a few years of age, with the CMV vaccine or pharmaceutical formulation thereof such that CMV infection can be completely prevented, or risk and/or severity of the infection in the subject can be substantially reduced. In some embodiments, the time for vaccination after birth can be within first one month, first five month, first year, first two years, first three years, first four years, first five years or any intervening period of the foregoing. If any child, adolescent or adult who is deemed or believed not to have been exposed to or infected with CMV previously, such subject can also be vaccinated with a CMV vaccine or pharmaceutical formulation thereof as described herein when desired or needed. In some embodiments, the subject can be a transplant patient and on such an occasion, CMV vaccination can be done before or after transplantation such that the transplant patient can be protected from CMV infection or reactivation. Also, a subject in need of CMV vaccination can be an individual having compromised immune system (assuming they have an immune system sufficient to respond to the vaccine) and the vaccination can be done any time in need.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. In some embodiments, the formulations according to the present disclosure, especially aiming to generate antibodies against CMV-infection, can include an adjuvant such as alum or monophosphoryl lipid A.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms depend on the particular compound employed and the effect to be achieved, and the pharmacodynamics associated with each compound in the individual as well as the target disease or condition and the stage thereof in the individual.

### Exemplary Embodiments

Other features of the invention will become apparent in the course of the following descriptions of exemplary embodiments, which are given for illustration of the invention and are not intended to be limiting thereof.
1. A composition comprising: at least one HLA-E ligand or a fragment, derivative or variant thereof capable of binding to HLA-E; and a pharmaceutically acceptable carrier, wherein the composition is capable of inducing an immune response.
2. The composition of embodiment 1, wherein the HLA-E ligand is capable of binding to a) a CD94-NKG2C receptor that is present on natural killer (NK) cells or T cells; or b) a T cell antigen receptor.
3. The composition of embodiment 1, wherein the HLA-E ligand binds with high affinity to a) a CD94-NKG2C receptor that is present on NK cells or T cells; or b) a T cell antigen receptor.
4. The composition of embodiment 2 or 3, wherein the HLA-E ligand is associated with HLA-E when binding to a) a CD94-NKG2C receptor that is present on NK cells or T cells; or b) a T cell antigen receptor.
5. The composition of any one of embodiments 1-4, further comprising a linker.
6. The composition of any one of embodiments 1-5, wherein the composition comprises more than one HLA-E ligand or a fragment, derivative or variant thereof.
7. The composition of any one of embodiments 1-6, further comprising: HLA-E or a fragment, derivative or variant thereof.
8. The composition of embodiment 7, wherein the HLA-E or the fragment, derivative or variant thereof is a soluble and secretory form.
9. The composition of any one of embodiments 1-8, wherein the immune response comprises an increase of NK cell-mediated killing or T cell-mediated killing of CMV-infected cells.
10. The composition of one of embodiments 1-9, wherein the immune response comprises proliferation of T cells and/or NK cells.
11. The composition of embodiment 9 or 10, wherein the T cells and/or NK cells express CD94-NKG2C receptor.
12. The composition of any one of embodiments 9-11, wherein the T cells and/or NK cells are specific for CMV-infected cells.
13. The composition of any one of embodiments 1-12, wherein the immune response comprises increase of T cells and/or NK cells that express CD94-NKG2C receptor.
14. The composition of any one of embodiments 1-13, wherein the immune response comprises killing of CMV-infected cells.
15. The composition of any one of embodiments 1-14, wherein the immune response comprises T cell-mediated killing of CMV-infected cells.
16. The composition of any one of embodiments 1-14, wherein the immune response comprises NK cell-mediated killing of CMV-infected cells.
17. The composition any one of embodiments 1-16, wherein the immune response is increased production or secretion of one or more cytokines.
18. The composition of embodiment 17, wherein the cytokine is interferon gamma.
19. The composition of any one of embodiments 7-18, wherein the HLA-E or the fragment, derivative or variant of HLA-E and the HLA-E ligand or the fragment, derivative or variant of HLA-E ligand do not substantially bind to inhibitory CD94-NKG2A receptor of NK cells or T cells.
20. The composition of any one of embodiments 1-19, wherein the composition is formulated as a Cytomegalovirus (CMV) vaccine.
21. The composition of any one of embodiments 1-20, wherein the HLA-E ligand or the fragment, derivative or variant thereof is selected from the group consisting of the sequences identified in Table 1 and Table 2 and sequences having at least 85% identity to the sequences identified in Table 1 and Table 2.
22. The composition of any one of embodiment 7 to 21, wherein the HLA-E or the fragment, derivative or variant thereof comprises the sequence of SEQ ID NO. 1, 2, or 3, or a variant thereof having at least 85% identity to the sequence of SEQ ID NO. 1, 2, or 3.
23. The composition of any one of embodiments 7-22, wherein the HLA-E or the fragment, derivative or variant of HLA-E and the HLA-E ligand or the fragment, derivative or variant of HLA-E ligand are covalently associated via a linker.
24. The composition of embodiment 23, wherein the linker comprises the sequence of a (G₄S)₃, (G₄S)₄ or (G₄S)₅.
25. A method of inducing an immune response in a subject, the method comprising: administering, to a subject, an effective amount of a composition comprising: at least one HLA-E ligand or a fragment, derivative or variant thereof capable of binding to HLA-E; and a pharmaceutically acceptable carrier.
26. The method of embodiment 25, wherein the subject has or is suspected of having CMV infection.
27. The method of embodiment 25, wherein the subject does not have CMV infection.
28. The method of any one of embodiments 25-27, wherein the subject is a child or an infant.
29. The method of any one of embodiments 25-27, wherein the subject is a woman prior to pregnancy.
30. The method of any one of embodiments 25-29, wherein the subject has a compromised immune system.
31. The method of embodiment 30, wherein the subject is a transplant patient.
32. The method of any one of embodiments 25-31, wherein the induction of immune response comprises expansion of NK cells or T cells.
33. The method of any one of embodiments 25-31, wherein induction of immune response comprises an increase of cells expressing NKG2C in the subject.
34. The method of embodiment 33, wherein the cells expressing NKG2C are NK cells and/or T cells.
35. The method of any one of embodiments 25-34, wherein the viral load of CMV is decreased.
36. A method of making a composition for inducing an immune response, the method comprising: formulating at least one HLA-E ligand or a fragment, derivative or variant thereof capable of binding to HLA-E and a pharmaceutically acceptable carrier in a form suitable for administration.
37. The method of embodiment 36, wherein HLA-E or a fragment, derivative or variant of HLA-E is formulated with the HLA-E ligand or the fragment, derivative or variant of HLA-E ligand capable of binding to HLA-E and the pharmaceutically acceptable carrier in a form suitable for administration.
38. A method of making a composition for inducing an immune response, the method comprising: introducing a vector sequence encoding a recombinant protein to mammalian cells, allowing expression of the recombinant protein, wherein the recombinant protein comprises at least one HLA-E ligand or a fragment, derivative or variant thereof capable of binding to HLA-E; isolating the expressed recombinant protein; and formulating the isolated recombinant protein and a pharmaceutically acceptable carrier in a form suitable for administration.
39. The method of embodiment 38, wherein the recombinant protein further comprises HLA-E or a fragment, derivative or variant thereof.
40. The method of embodiment 39, wherein the recombinant protein further comprises a linker covalently associating the HLA-E ligand or the fragment, derivative or variant of HLA-E ligand capable of binding to HLA-E and the HLA-E or the fragment, derivative or variant of HLA-E.
41. The method of any one of embodiments 36-40, wherein the HLA-E ligand or the fragment thereof is identified via a method comprising: contacting each of CMV-infected cell extract and CMV-uninfected cell extract with a plurality of HLA-E or a fragment, derivative or variant thereof that is immobilized on a substrate; allowing molecules in each of the cell extracts to bind to the plurality of the immobilized HLA-E or the fragment, derivative or variant thereof; collecting the molecules in each of the cell extracts that bind to the plurality of the immobilized HLA-E or the fragment, derivative or variant thereof; comparing the collected molecules from each of the cell extracts to identify molecules that are enriched in the CMV-infected cell extract as compared to the CMV-uninfected cell extract; and determining the sequence of the enriched molecules.
42. The method of any one of embodiments 36-40, wherein the HLA-E ligand or the fragment thereof is identified via a method comprising: contacting each of CMV-infected cell extract and CMV-uninfected cell extract with a plurality of HLA-E or a fragment, derivative or variant thereof that is immobilized on a substrate; allowing molecules in each of the cell extracts to bind to the plurality of the immobilized HLA-E or the fragment, derivative or variant thereof; collecting the molecules in each of the cell extracts that bind to the plurality of the immobilized HLA-E or the fragment, derivative or variant thereof; comparing the collected molecules from each of the cell extracts to identify molecules that are enriched in the CMV-infected cell extract as compared to the CMV-uninfected cell extract; and determining the sequence of the enriched molecules.
43. A method of inducing an adaptive immune response in a subject in need thereof, the method comprising: administering, to the subject, antibodies specific to HLA-E or a fragment, derivative or variant of HLA-E and/or a HLA-E ligand or a fragment, derivative or variant of HLA-E ligand that is capable of binding to HLA-E.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

In one aspect, a series of experiments are conducted to identify physiologic ligands of HLA-E protein that are recognized with high specificity by the activating CD94-NKG2C receptor that allows human NK cells to specifically respond to CMV infection. The following provides details of certain exemplary methods and materials that can be used in the experiments.

### Cell Culture and Infection

U-373 MG cells were stably infected with a soluble HLA-E*01:03 construct (SEQ ID NO: 3) and demonstrated secretion of sHLA-E by uninfected and CMV-infected cells. sHLA-E*01:03-transfected U-373MG cells can be expanded in bioreactor units until 25 mg of sHLA-E*01:03 is obtained from uninfected cells. Cultures then can be infected at an MOI of 3 with AD169 strain CMV. Cells can be monitored daily for percentage of infected cells by staining with anti-gB CMV mAb (Virusys Corp) (100% of cells can be infected at this MOI). Secretion of sHLA-E*01:03 during infection can be monitored by ELISA until ≥25 mg is obtained for comparison to HLA-E from uninfected cells.

### HLA-E Protein Isolation and Purification

Twenty-five mg of sHLA-E*01:03 from CMV-infected and uninfected cells can be collected and passed over 50 mL cyanogen bromide-activated Sepharose fast flow columns coupled to anti-VLDLr mAb. HLA-E/peptide complexes bound to the column can be washed with 20 mM sodium phosphate buffer and eluted using 0.2 N acetic acid, pH 2.7, in 5 mL fractions. Peptide-containing fractions are combined, brought to 10% glacial acetic acid, and then heated to 76° C. Pooled peptides are loaded into a model 8050 Millipore stirred cell ultrafiltration device containing a 3 kDa cut-off regenerated cellulose membrane. Peptides that flow through the membrane are collected in 50 mL conical centrifuge tubes, flash frozen in liquid nitrogen, and lyophilized. Peptides are resuspended in 10% acetic acid in a final volume of 1 mL from uninfected cells and CMV-infected cells. Separate peptide purifications are performed from uninfected and infected cells using identical conditions.

### Peptide Analysis by Mass Spectrometry

High and low pH two-dimensional RP-HPLC precedes mass spectrometric ligand characterization. A mass spectrometer (e.g. ABSCIEX 5600) can be used such that two-dimensional LC-MS1 and LC-MS2 enable efficient separation and sequencing of HLA-E-eluted ligands. HLA-E HPLC peptide fractions from uninfected and CMV-infected cells can be solubilized in 1:1 water: dimethylformamide, injected into a Jupiter Proteo reverse phase 2 mm column, and eluted at high pH conditions (pH 10) with a gradient of 2-10% acetonitrile in water in 2 minutes and then 10-60% in 60 minutes. Twenty peptide-rich fractions can be collected along the gradient, dried by vacuum centrifugation, solubilized in a second dimension solvent A (0.1% formic acid, 2% acetonitrile, and 98% water), and then placed into the high-throughput autosampler of an Eksigent nanoLC400 U-HPLC system (AB Sciex). One-tenth of each first dimension high pH HPLC sample can be run on a reverse-phase nano-HPLC column equilibrated at pH 2 whereby peptides are eluted using a program with dual linear gradients. The second-dimension HPLC column effluent is connected to a nanospray III ion source of the AB Sciex 5600 quadrupole-TOF mass spectrometer in order to generate LC/MS ion maps and parent ion MS/MS fragmentation spectra. The 20 second-dimension LC/MS1 spectra can be comparatively analyzed and candidate ligand sequences can be identified.

### Tandem Mass Spectrometric Analysis

Because of the nature of HLA peptides, a multi-layered complementary approach can be taken to resolve MS2 fragmentation data, utilizing a combined application of the current algorithms MASCOT (Matrix Science), PEAKS (Bioinformatic Solutions), and ProteinPilot (AB Sciex). Using this combined approach it is possible to sequence >30,000 classical HLA-derived peptides with high confidence at 1% false discovery rate, including any peptides that are post-translational modified (approximately >20% of the ligands).

CMV infection can alter the HLA-E protein repertoire with the induction of viral peptides, as well as the generation of novel host-derived human peptides resulting from cellular stress caused by viral infection. The advantage of isolating peptides from soluble HLA-E is that the sHLA-E construct can be constitutively expressed to provide high amounts of secreted protein whereas endogenous HLA-E is expressed at low amounts on the cell surface. Moreover, viable cells continuously produce sHLA-E whereas detergent solubilization destroys the HLA-producing cells. Detergents used to isolate membrane proteins produce low amounts of HLA in complex mixtures that require extensive purification with detergent residues inappropriate for MS analysis. Soluble HLA-E provides a ready tool for the systematic identification of viral and host ligands distinct to infected cells.

### Example 2

CMV-infection can induce host- or virus-encoded peptides loading HLA-E that might generate high affinity ligands for the CD94-NKG2C receptor. To identify CMV-induced HLA-E peptide complexes that preferentially bind to the activating CD94-NKG2C receptor, the experiments can be conducted so as to identify novel host-derived or viral peptides bound to HLA-E in CMV-infected cells. By comparing the repertoire of peptides from uninfected versus infected cells peptides that are unique to CMV-infected cells and host-derived peptides, which are over-represented in infected cells for their ability to affect the binding of HLA-E to either CD94-NKG2A or CD94-NKG2C can be selected. Peptides corresponding to candidate HLA-E-eluted peptides can be synthesized in small scale and sequenced to confirm fidelity. Peptides (0-300 μM titration) can be incubated overnight at 37° C. or 25° C. with TAP-deficient RMA-S cells that are stably transfected with human HLA-E and human β2-microglobulin, and then stained for HLA-E using a FITC-anti-HLA class I mAb and analyzed by flow cytometry. This assay can validate that the candidate peptides can stabilize cell surface expression of HLA-E. As a positive control there can be a synthetic peptide corresponding to the signal sequence of HLA-G (VMAPRTLFL), which allows HLA-E to bind both CD94-NKG2A and CD94-NKG2C (albeit with low affinity). For peptides that stabilize HLA-E on the surface of the HLA-E RMA-S transfectants, these transfectants with CD94-NKG2A or CD94-NKG2C fusion proteins can be stained to assess differential binding to these receptors. There can be an expression vector that contains a CD8 leader, human IgG1 Fc, and a linker into which cDNA encoding the extracellular domain of type II membrane proteins is inserted. Because CD94 can form homodimers, in addition to heterodimers with NKG2A or NKG2C, a Flag tag was introduced onto the N-terminus of NKG2A and NKG2C-Fc cDNA constructs. 293T cells are co-transfected with the CD94-Fc and NKG2A-Fc-Flag or NKG2C-Fc-Flag vectors and fusion proteins purified by using protein A-Sepharose. Although homodimers of CD94, NKG2A, or NKG2C do not bind HLA-E, there can be affinity purification of the fusion proteins on an anti-CD94 mAb-Sepharose column and subsequently purify the heterodimers on an anti-Flag mAb Sepharose column. CD94-NKG2A-Fc and CD94-NKG2C-Fc can be used to stain the HLA-E RMA-S cells loaded with candidate peptides, followed by detection using flow cytometry. HLA-E RMA-S cells loaded with the synthetic HLA-G leader peptide can be used as a positive control. Should the dimeric CD94-NKG2A-Fc and CD94-NKG2C-Fc fusion proteins be sub-optimal for binding to peptide-loaded HLA-E transfected RMA-S cells, the avidity of the staining reagent can be increased by making pre-formed complexes of phycoerythrin-conjugated anti-human IgG and the fusion proteins.

The relative affinity of CD94-NKG2A and CD94-NKG2C fusion protein binding to the HLA-E RMA-S cells loaded with the candidate peptides identified in CMV-infected cells can be determined to estimate relative affinities of TcR for H-2 ligands. Briefly, H-2-peptide tetramers can be used to stain TcR transgenic cells, and then a blocking anti-H-2 mAb can be added to prevent re-binding of any dissociated tetramer. Cells may be assayed by flow cytometry over a time course for loss of tetramer staining. This assay is thus able to distinguish TcR with faster dissociation rates versus TcR with stronger binding activity. The candidate peptide-loaded HLA-E RMA-S cells can be stained at room temperature with saturating amounts of the CD94-NKG2A or CD94-NKG2C Fc, wash the cells, and then add control Ig or neutralizing anti-CD94 mAb. Aliquots of cells held at 4° C. can be removed over a time course, stained with anti-IgG 2n d step, and cells can be analyzed by flow cytometry. Conditions (time, reagent concentrations, and temperature) can be optimized using HLA-E RMA-S cells loaded with HLA-G leader peptide because the affinity of this ligand for CD94-NKG2A and CD94-NKG2C has been established.

Viral or host-derived peptides that preferentially bind CD94-NKG2C Fc can be assayed for their ability to bind and activate primary human NKG2C+NK cells. Human NKG2C+NK cells can be co-cultured with the HLA-E RMA-S transfectants or human HLA-E-transfected 721.221 cells loaded with candidate peptides and assayed for their ability to kill these targets, secrete interferon-gamma, and proliferate using assays. Blocking antibodies to CD94 and NKG2C can be used to confirm the specificity of functional responses induced by these peptide-loaded cells. For viral peptide candidates, a comprehensive library of mutant CMV strains (e.g. from Fenyong Liucan) so that mutant stains lacking the viral gene encoding the candidate peptide for their ability to stimulate primary NK cells expressing the NKG2C receptor can be tested. Restoration of the candidate viral gene in the mutant stain can validate putative ligands. Table 1 shows some selected viral peptides identified as candidates for HLA-E ligands.

TABLE 1
HLA-E ligands

| | | | | | Found | | | | | | | | | | |
|---|---|---|---|---|---|---|---|---|---|---|---|---|---|---|---|
| | | | | | in | | | | | | | | | | |
| Uniprot Protein | HCMV Source | Gene | Peptide Sequence | Length | NCBI | PEAKS | | delta | | | | # | Start | End | PTM |
| Accession | Protein | symbol | (SEQ ID NO) | (aa) | Virus | score | Mass | ppm | m/z | z | RT | Spec | AA | AA | Present |
| P09715\|IRS1_HCMVA | Protein IRS1 | IRS1 | PTYDELPSRPPQ (4) | 12 | N | 63.18 | 1399 | 2.5 | 700 | 2 | 20 | 3 | 730 | 741 | N |
| P16755\|UL13_HCMVA | Uncharacterized | UL13 | LDIVEEDEWLR (5) | 11 | Y | 71.1 | 1458 | 7.7 | 730 | 2 | 54 | 1 | 37 | 47 | Y |
| | protein UL13 | | | | | | | | | | | | | | |
| P16845\|UL22A_HCMVA | glycoprotein UL22A | UL22A | APSQKSKRSVTVEQPS | 20 | Y | 67.51 | 2102 | 2.9 | 527 | 4 | 9.5 | 20 | 21 | 40 | N |
| | | | TSAD (6) | | | | | | | | | | | | |
| P16845\|UL22A_HCMVA | glycoprotein UL22A | UL2ZA | APSQKSKRSVTVEQPS | 23 | Y | 46.1 | 2360 | 0.5 | 591 | 4 | 10 | 1 | 21 | 43 | N |
| | | | TSADGSN (7) | | | | | | | | | | | | |
| P16845\|UL22A_HCMVA | glycoprotein UL22A | UL22A | SQKSKRSVTVEQPSTS | 18 | Y | 53.42 | 1934 | 5.4 | 485 | 4 | 9.7 | 3 | 23 | 40 | N |
| | | | AD (8) | | | | | | | | | | | | |
| P16845\|UL22A_HCMVA | glycoprotein UL22A | UL22A | RSVTVEQPSTSAD (9) | 13 | Y | 54.16 | 1376 | 2.6 | 689 | 2 | 12 | 2 | 28 | 40 | N |
| P16845\|UL22A_HCMVA | glycoprotein UL22A | UL22A | SVTVEQPSTSAD (10) | 12 | Y | 72.51 | 1220 | 7.5 | 611 | 2 | 14 | 11 | 29 | 40 | N |
| P16845\|UL22A_HCMVA | glycoprotein UL22A | UL22A | SVTVEQPSTSADGSN | 15 | Y | 53.27 | 1478 | 8.3 | 740 | 2 | 14 | 1 | 29 | 43 | N |
| | | | (11) | | | | | | | | | | | | |
| P16845\|UL22A_HCMVA | glycoprotein UL22A | UL22A | SVTVEQPSTSA (12) | 11 | Y | 49.73 | 1105 | −1.2 | 553 | 2 | 15 | 2 | 29 | 39 | N |
| P16845\|UL22A_HCMVA | glycoprotein UL22A | UL22A | VTVEQPSTSAD (13) | 11 | Y | 64.07 | 1133 | 1.5 | 567 | 2 | 14 | 3 | 30 | 40 | N |
| P16845\|UL22A_HCMVA | glycoprotein UL22A | UL22A | GDEDYSGEYDVL (14) | 12 | Y | 59.25 | 1361 | 12.5 | 681 | 2 | 32 | 3 | 61 | 72 | N |
| P16845\|UL22A_HCMVA | glycoprotein UL22A | UL22A | LITDGDGSEHQQP (15) | 13 | Y | 44.65 | 1396 | 9.1 | 699 | 2 | 13 | 4 | 72 | 84 | N |
| P16845\|UL22A_HCMVA | glycoprotein UL22A | UL22A | EHKENQAKENEKKIQ | 15 | Y | 47.79 | 1853 | 13.9 | 464 | 4 | 10 | 1 | 89 | 103 | Y |
| | | | (16) | | | | | | | | | | | | |
| P16794\|UL53_HCMVA | Virion egress | UL31 | SSVSGVRTPR (17) | 10 | N | 38.96 | 1087 | 3.1 | 544 | 2 | 12 | 2 | 2 | 11 | Y |
| | protein UL31 | | | | | | | | | | | | | | |
| P08318\|PP150_HCMVA | tegument protein | UL32 | SLQFIGLQRRD (18) | 11 | N | 50.99 | 1374 | 2.4 | 688 | 2 | 34 | 3 | 2 | 12 | Y |
| | pp150 | | | | | | | | | | | | | | |
| P08318\|PP150_HCMVA | tegument protein | UL32 | TVAFDLSSPQK (19) | 11 | Y | 77.17 | 1192 | 2.8 | 597 | 2 | 23 | 2 | 1001 | 1011 | N |
| | pp150 | | | | | | | | | | | | | | |
| P16766\|UL35_HCMVA | tegument protein | UL35 | AQGSRAPSGPPLPVLP | 18 | N | 67.4 | 1799 | 2.8 | 601 | 3 | 39 | 1 | 2 | 19 | Y |
| | UL35 | | VD (20) | | | | | | | | | | | | |
| P16766\|UL35_HCMVA | tegument protein | UL35 | ARERGEFGDEDEEQE | 21 | N | 71.98 | 2463 | 28 | 617 | 4 | 13 | 1 | 351 | 371 | N |
| | UL35 | | NDGEPR (21) | | | | | | | | | | | | |
| P16766\|UL35_HCMVA | tegument protein | UL35 | ARERGEFGDEDEEQE | 17 | N | 49.16 | 2024 | −4.4 | 676 | 3 | 12 | 1 | 351 | 367 | N |
| | UL3S | | ND (22) | | | | | | | | | | | | |
| P16766\|UL35_HCMVA | tegument protein | UL35 | GEPREAQLDLEAD (23) | 13 | N | 66.74 | 1442 | 9.7 | 722 | 2 | 21 | 1 | 368 | 380 | N |
| | UL35 | | | | | | | | | | | | | | |
| P16766\|UL35_HCMVA | tegument protein | UL35 | EAQLDLEAD (24) | 9 | N | 60.92 | 1002 | 5 | 502 | 2 | 23 | 5 | 372 | 380 | N |
| | UL35 | | | | | | | | | | | | | | |
| P16766\|UL35_HCMVA | tegument protein | UL35 | RAPLGQESEPEITEH | 15 | Y | 92.05 | 1692 | 7.7 | 565 | 3 | 16 | 2 | 470 | 484 | N |
| | UL35 | | (25) | | | | | | | | | | | | |
| P16766\|UL35_HCMVA | tegument protein | UL35 | RAPLGQESEPEITEHR | 16 | Y | 70.78 | 1848 | −2 | 617 | 3 | 14 | 2 | 470 | 485 | N |
| | UL35 | | (26) | | | | | | | | | | | | |
| P16766\|UL35_HCMVA | tegument protein | UL35 | PRDDLAENLRHL (27) | 12 | N | 90.49 | 1448 | 4.8 | 363 | 4 | 23 | 18 | 629 | 640 | N |
| | UL35 | | | | | | | | | | | | | | |
| P16784\|UL37_HCMVA | Capsid assembly | UL37 | PPAGSTSVSLPPASP | 15 | N | 73.33 | 1364 | 3 | 683 | 2 | 22 | 100 | 969 | 983 | N |
| | protein UL37 | | (28) | | | | | | | | | | | | |
| P16780\|UL40_HCMVA | Protein UL40 | UL40 | VMAPRTLIL (29) | 9 | N | 50.21 | 1013 | 5.6 | 507 | 2 | 31 | 156 | 15 | 23 | N |
| P16780\|UL40_HCMVA | Protein UL40 | UL40 | VMAPRTLI (30) | 8 | N | 44.21 | 915.5 | 1 | 459 | 2 | 20 | 90 | 15 | 22 | Y |
| P16780\|UL40_HCMVA | Protein UL40 | UL40 | VMAPRTL (31) | 7 | N | 38.05 | 786.4 | 1.7 | 394 | 2 | 16 | 36 | 15 | 21 | N |
| P16815\|UL42_HCMVA | Uncharacterized | UL42 | TVVINRDSANITTGTQ | 20 | N | 50.85 | 1991 | 8 | 665 | 3 | 18 | 3 | 106 | 125 | N |
| | protein UL42 | | ASSG (32) | | | | | | | | | | | | |
| P16790\|VPAP_HCMVA | DNA polymerase | UL44 | TINNSTPLL (33) | 9 | N | 42.3 | 971.5 | 4.2 | 487 | 2 | 28 | 2 | 79 | 87 | N |
| | processivity | | | | | | | | | | | | | | |
| | subunit | | | | | | | | | | | | | | |
| P16790\|VPAP_HCMVA | DNA polymerase | UL44 | FGVVADLLKWIGPHTR | 17 | N | 43.26 | 1929 | 2.1 | 483 | 4 | 26 | 4 | 150 | 166 | N |
| | processivity | | V (34) | | | | | | | | | | | | |
| | subunit | | | | | | | | | | | | | | |
| P16790\|VPAP_HCMVA | DNA polymerase | UL44 | PFDKNYVGNSGKSRG | 34 | N | 87.47 | 3191 | 19.6 | 799 | 4 | 22 | 1 | 277 | 310 | Y |
| | processivity | | GGGGGGSLSSLANAG | | | | | | | | | | | | |
| | subunit | | GLHD (35) | | | | | | | | | | | | |
| P16790\|VPAP_HCMVA | DNA polymerase | UL44 | PFDKNYVGNSGK (36) | 12 | N | 85.67 | 1325 | 3.1 | 443 | 3 | 12 | 9 | 277 | 288 | N |
| | processivity | | | | | | | | | | | | | | |
| | subunit | | | | | | | | | | | | | | |
| P16790\|VPAP_HCMVA | DNA polymerase | UL44 | PFDKNYVGNSG (37) | 11 | N | 83.7 | 1197 | 1.9 | 599 | 2 | 14 | 6 | 277 | 287 | N |
| | processivity | | | | | | | | | | | | | | |
| | subunit | | | | | | | | | | | | | | |
| P16790\|VPAP_HCMVA | DNA polymerase | UL44 | PFDKNYVGNSGKSRG | 35 | Y | 74.17 | 3305 | 8.7 | 827 | 4 | 21 | 1 | 277 | 311 | N |
| | processivity | | GGGGGGSLSSLANAG | | | | | | | | | | | | |
| | subunit | | GLHDD (38) | | | | | | | | | | | | |
| P16790\|VPAP_HCMVA | DNA polymerase | UL44 | PFDKNYVGNSGKS (39) | 13 | N | 61.26 | 1412 | 4.1 | 472 | 3 | 12 | 1 | 277 | 289 | N |
| | processivity | | | | | | | | | | | | | | |
| | subunit | | | | | | | | | | | | | | |
| P16790\|VPAP_HCMVA | DNA polymerase | UL44 | PFDKNYVGNSGKSRG | 29 | N | 45.75 | 2711 | 12.4 | 679 | 4 | 20 | 1 | 277 | 305 | Y |
| | processivity | | GGGGGGSLSSLANA | | | | | | | | | | | | |
| | subunit | | (40) | | | | | | | | | | | | |
| P16790\|VPAP_HCMVA | DNA polymerase | UL44 | PFDKNYVGNSGKSRG | 26 | N | 44.14 | 2454 | −0.1 | 615 | 4 | 19 | 4 | 277 | 302 | N |
| | processivity | | GGGGGGSLSSL (41) | | | | | | | | | | | | |
| | subunit | | | | | | | | | | | | | | |
| P16790\|VPAP_HCMVA | DNA polymerase | UL44 | SRGGGGGGGSLSSLA | 23 | Y | 64.42 | 1998 | 5 | 667 | 3 | 22 | 3 | 289 | 311 | N |
| | processivity | | NAGGLHDD (42) | | | | | | | | | | | | |
| | subunit | | | | | | | | | | | | | | |
| P16790\|VPAP_HCMVA | DNA polymerase | UL44 | GPGLDNDLMNEPMG | 29 | N | 43.31 | 2664 | −18.4 | 667 | 4 | 28 | 1 | 312 | 340 | Y |
| | processivity | | LGGLGGGGGGGGKK | | | | | | | | | | | | |
| | subunit | | H (43) | | | | | | | | | | | | |
| P16790\|VPAP_HCMVA | DNA polymerase | UL44 | SEDSVTFEFVPNTKKQ | 16 | N | 47.87 | 1855 | 24.6 | 619 | 3 | 27 | 2 | 415 | 430 | N |
| | processivity | | (44) | | | | | | | | | | | | |
| | subunit | | | | | | | | | | | | | | |
| P16790\|VPAP_HCMVA | DNA polymerase | UL44 | SVTFEFVPNTK (45) | 11 | Y | 74.64 | 1268 | −6 | 635 | 2 | 30 | 2 | 418 | 428 | N |
| | processivity | | | | | | | | | | | | | | |
| | subunit | | | | | | | | | | | | | | |
| P16790\|VPAP_HCMVA | DNA polymerase | UL44 | VTFEFVPNTK (46) | 10 | N | 55.97 | 1181 | 0 | 591 | 2 | 30 | 1 | 419 | 428 | N |
| | processivity | | | | | | | | | | | | | | |
| | subunit | | | | | | | | | | | | | | |
| P16783\|VP19_HCMVA | Triplex capsid | UL46 | MDARAVAKRPRD (47) | 12 | N | 48.61 | 1427 | 2.5 | 477 | 3 | 12 | 2 | 1 | 12 | Y |
| | protein VP19C | | | | | | | | | | | | | | |
| P16783\|VP19_HCMVA | Triplex capsid | UL46 | NFSVELGDFREFV (48) | 13 | Y | 51.93 | 1558 | −1.5 | 780 | 2 | 49 | 1 | 278 | 290 | N |
| | protein VP19C | | | | | | | | | | | | | | |
| Q7M6N6\|UL48A_HCMVA | small capsid | UL48A | SNTAPGPTVANKRD | 14 | Y | 66.04 | 1469 | 6.5 | 735 | 2 | 13 | 14 | 2 | 15 | Y |
| | protein UL48A | | (49) | | | | | | | | | | | | |
| Q7MGN6\|UL48A_HCMVA | small capsid | UL48A | SNTAPGPTVAN (50) | 11 | N | 50.23 | 1070 | 0.4 | 536 | 2 | 16 | 3 | 2 | 12 | Y |
| | protein UL48A | | | | | | | | | | | | | | |
| P16793\|UL52_HCMVA | Packaging protein | UL52 | PTYVIDKYV (51) | 9 | N | 64.14 | 1097 | 3.4 | 549 | 2 | 29 | 2 | 660 | 668 | N |
| | UL32 | | | | | | | | | | | | | | |
| P17147\|DNBI_HCMVA | Major DNA-binding | UL57 | PVTGEDTFSAHGKSD | 15 | N | 75.41 | 1547 | 7.3 | 517 | 3 | 15 | 1 | 615 | 629 | N |
| | protein | | (52) | | | | | | | | | | | | |
| P17147\|DNBI_HCMVA | Major DNA-binding | UL57 | QNVALITAT (53) | 9 | N | 42.46 | 913.5 | 6.9 | 458 | 2 | 12 | 3 | 696 | 704 | Y |
| | protein | | | | | | | | | | | | | | |
| P17147\|DNBI_HCMVA | Major DNA-binding | UL57 | EAGGVGGSSGGGGGS | 25 | Y | 119.5 | 2183 | 10.2 | 547 | 4 | 16 | 3 | 1211 | 1235 | N |
| | protein | | GLLPAKRSRL (54) | | | | | | | | | | | | |
| P17147\|DNBI_HCMVA | Major DNA-binding | UL57 | EAGGVGGSSGGGGGS | 23 | Y | 78.06 | 1914 | 6.1 | 639 | 3 | 14 | 1 | 1211 | 1233 | N |
| | protein | | GLLPAKRS (55) | | | | | | | | | | | | |
| P17147\|DNBI_HCMVA | Major DNA-binding | UL57 | EAGGVGGSSGGGGGS | 22 | Y | 67.64 | 1827 | 0.7 | 610 | 3 | 15 | 3 | 1211 | 1232 | N |
| | protein | | GLLPAKR (56) | | | | | | | | | | | | |
| P16749\|ICP27_HCMVA | mRNA export | UL69 | PATLTAYDK (57) | 9 | N | 43.69 | 978.5 | 3.3 | 490 | 2 | 15 | 2 | 572 | 580 | N |
| | factor ICP27 | | | | | | | | | | | | | | |
| P16749\|ICP27_HCMVA | mRNA export | UL69 | QPPPPPPPP (58) | 9 | N | 55.63 | 922.5 | 31.4 | 462 | 2 | 12 | 2 | 706 | 714 | N |
| | factor ICP27 | | | | | | | | | | | | | | |
| P06726\|PP71_HCMVA | tegument protein | UL82 | EQDRLLVDL (59) | 9 | N | 49.37 | 1143 | −2.7 | 572 | 2 | 55 | 15 | 98 | 106 | Y |
| | pp71 | | | | | | | | | | | | | | |
| P06726\|PP71_HCMVA | tegument protein | UL82 | PNTYIHKTETD (60) | 11 | N | 77.19 | 1318 | 2.6 | 440 | 3 | 11 | 2 | 221 | 231 | N |
| | pp71 | | | | | | | | | | | | | | |
| P06726\|PP71_HCMVA | tegument protein | UL82 | DEDDLSSTPTPTPL | 14 | Y | 87.48 | 1487 | 3.9 | 344 | Z | 28 | 3 | 426 | 439 | N |
| | pp71 | | (61) | | | | | | | | | | | | |
| P06726\|PP71_HCMVA | tegument protein | UL82 | LSSTPTPTPL (62) | 10 | Y | 58.82 | 1013 | 3.2 | 507 | 2 | 22 | 6 | 430 | 439 | N |
| | pp71 | | | | | | | | | | | | | | |
| P06726\|PP71_HCMVA | tegument protein | UL82 | MDGDVRTAADISSTER | 16 | N | 40.82 | 1723 | 9.9 | 575 | 3 | 21 | 1 | 519 | 534 | Y |
| | pp73 | | (63) | | | | | | | | | | | | |
| P06726\|PP71_HCMVA | tegument protein | UL82 | GDVRTAADISSTLRSV | 34 | Y | 78.43 | 3438 | 11.3 | 860 | 4 | 27 | 4 | 521 | 554 | N |
| | pp71 | | PAPRPSPISTASTSSTP | | | | | | | | | | | | |
| | | | R (64) | | | | | | | | | | | | |
| P06726\|PP71_HCMVA | tegument protein | UL82 | GDVRTAADISSTERSY | 27 | N | 53.56 | 2721 | 0.1 | 681 | 4 | 29 | 3 | 521 | 547 | N |
| | pp71 | | PAPRPSPISTA (65) | | | | | | | | | | | | |
| P06725\|PP71_HCMVA | tegument protein | UL82 | VRTAADISSTLRSVPAP | 21 | Y | 72.2 | 2177 | 8.6 | 545 | 4 | 24 | 3 | 523 | 543 | N |
| | pp71 | | RPSP (66) | | | | | | | | | | | | |
| P06726\|PP71_HCMVA | tegument protein | UL82 | VRTAADISSTLR (67) | 12 | N | 61.9 | 1289 | −2.6 | 431 | 3 | 16 | 2 | 523 | 534 | N |
| | pp71 | | | | | | | | | | | | | | |
| P06726\|PP71_HCMVA | tegument protein | U182 | VRTAADISSTLRSVPAP | 25 | N | 52.21 | 2549 | 5.1 | 638 | 4 | 27 | 3 | 523 | 547 | N |
| | pp71 | | RPSPISTA (68) | | | | | | | | | | | | |
| P06726\|PP71_HCMVA | tegument protein | U182 | ISSTLRSVPAPRPSPIST | 19 | Y | 72.23 | 1936 | 1.2 | 646 | 3 | 20 | 4 | 529 | 547 | N |
| | pp71 | | A (69) | | | | | | | | | | | | |
| P06726\|PP71_HCMVA | tegument protein | UL82 | SVPAPRPSPISTASTSS | 20 | Y | 95.99 | 1995 | −0.4 | 666 | 3 | 14 | 4 | 535 | 554 | N |
| | pp71 | | TPR (70) | | | | | | | | | | | | |
| P06726\|PP71_HCMVA | tegument protein | UL82 | SVPAPRPSPISTA (71) | 13 | Y | 64.41 | 1279 | 2.1 | 640 | 2 | 16 | 6 | 535 | 547 | N |
| | pp71 | | | | | | | | | | | | | | |
| P06726\|PP71_HCMVA | tegument protein | UL82 | SVPAPRPSPISTAST | 15 | Y | 56.78 | 1467 | 3 | 734 | 2 | 16 | 3 | $35 | 549 | N |
| | pp/1 | | (72) | | | | | | | | | | | | |
| P06726\|PP71_HCMVA | tegument protein | UL82 | SVPAPRPSPIST (73) | 12 | Y | 54.05 | 1208 | −2.5 | 605 | 2 | 16 | 2 | 535 | 546 | N |
| | pp71 | | | | | | | | | | | | | | |
| P06725\|PP65_HCMVA | tegument protein | UL83 | MISVLGPISGHVLKAV | 21 | N | 94.81 | 2240 | 5.3 | 748 | 3 | 52 | 3 | 11 | 31 | Y |
| | pp65 | | FSRGD (74) | | | | | | | | | | | | |
| P06725\|PP65_HCMVA | tegument protein | UL83 | MISVLGPISGHVLK | 14 | Y | 73.61 | 1466 | 4.4 | 490 | 3 | 30 | 3 | 11 | 24 | Y |
| | pp6S | | (75) | | | | | | | | | | | | |
| P06725\|PP65_HCMVA | tegument protein | U183 | SVLGPISGHVLK (76) | 12 | Y | 62.63 | 1206 | −2.4 | 403 | 3 | 24 | 4 | 13 | 24 | N |
| | pp65 | | | | | | | | | | | | | | |
| P06725\|PP65_HCMVA | tegument protein | U183 | SVLGPISGHV (77) | 10 | Y | 45.98 | 964.5 | 5.1 | 483 | 2 | 25 | 4 | 13 | 22 | N |
| | pp55 | | | | | | | | | | | | | | |
| P06725\|PP65_HCMVA | tegument protein | UL83 | VLGPISGHV (78) | 9 | N | 40.63 | 877.5 | 3.6 | 440 | 2 | 21 | 1 | 14 | 22 | N |
| | pp65 | | | | | | | | | | | | | | |
| P06725\|PP65_HCMVA | tegument protein | UL83 | VLGPISGHVLK (79) | 11 | N | 39.04 | 1119 | −2.3 | 374 | 3 | 19 | 1 | 14 | 24 | N |
| | pp65 | | | | | | | | | | | | | | |
| P06725\|PP65_HCMVA | tegument protein | UL83 | SEVENVSVNVHNPTG | 16 | Y | 70.9 | 1737 | 3.3 | 580 | 3 | 17 | 3 | 85 | 100 | N |
| | pp65 | | R (80) | | | | | | | | | | | | |
| P06725\|PP65_HCMVA | tegument protein | UL83 | SEVENVSVNVHNPTG | 15 | Y | 53.73 | 1581 | 9.3 | 791 | 2 | 19 | 2 | 85 | 99 | N |
| | pp65 | | (81) | | | | | | | | | | | | |
| P06725\|PP65_HCMVA | tegument protein | UL83 | HRHLPVAD (82) | 8 | N | 54.31 | 943.5 | 3.9 | 473 | 2 | 10 | 3 | 139 | 146 | |
| | pp65 | | | | | | | | | | | | | | |
| P06725\|PP65_HCMVA | tegument protein | UL83 | TSAFVFPTK (83) | 9 | Y | 79.49 | 996.5 | −5 | 499 | 2 | 26 | 2 | 183 | 191 | N |
| | pp65 | | | | | | | | | | | | | | |
| P06725\|PP65_HCMVA | tegument protein | UL83 | WDRHDEGAAQGDDD | 31 | Y | 131.9 | 3477 | 3.4 | 870 | 4 | 27 | 2 | 385 | 415 | ? |
| | pp65 | | VWTSGSDSDEELVTTE | | | | | | | | | | | | |
| | | | (84) | | | | | | | | | | | | |
| P06725\|PP65_HCMVA | tegument protein | UL83 | AQGDDDVWTSGSDS | 23 | Y | 110.6 | 2511 | 6.8 | 838 | 3 | 29 | 3 | 393 | 415 | N |
| | pp65 | | DEELVTTER (85) | | | | | | | | | | | | |
| P06725\|PP65_HCMVA | tegument protein | UL83 | GDDDVWTSGSDSDEE | 23 | Y | 107.7 | 2312 | 9.4 | 772 | 3 | 30 | 1 | 395 | 415 | N |
| | pp65 | | LVTTER (86) | | | | | | | | | | | | |
| P06725\|PP65_HCMVA | tegument protein | UL83 | DDDVWTSGSDSDEEL | 20 | Y | 113.8 | 2255 | 2.8 | 753 | 3 | 30 | 3 | 396 | 415 | N |
| | pp65 | | VTTER (87) | | | | | | | | | | | | |
| P06725\|PP65_HCMVA | tegument protein | UL83 | DVWTSGSDSDEELVT | 18 | Y | 101.2 | 2025 | 10.8 | 675 | 3 | 28 | 5 | 398 | 415 | N |
| | pp65 | | TER (88) | | | | | | | | | | | | |
| P06725\|PP65_HCMVA | tegument protein | UL83 | VWTSGSDSDEELVITTE | 17 | Y | 107.3 | 1910 | 6.4 | 638 | 3 | 26 | 5 | 394 | 415 | N |
| | pp65 | | (89) | | | | | | | | | | | | |
| P06725\|PP65_HCMVA | tegument protein | UL83 | VWTSGSDSDEELVITTE | 21 | Y | 105.6 | 2392 | 1.6 | 599 | 4 | 20 | 2 | 399 | 419 | N |
| | pp65 | | RKTPR (90) | | | | | | | | | | | | |
| P06725\|PP65_HCMVA | tegument protein | UL83 | VWTSGSDSDEELVTTE | 16 | Y | 64.71 | 1754 | 10.4 | 878 | 2 | 28 | 3 | 399 | 414 | N |
| | pp65 | | (91) | | | | | | | | | | | | |
| P06725\|PP65_HCMVA | tegument protein | UL83 | VWTSGSDSDEELVTTE | 18 | N | 41.44 | 2038 | −1.6 | 680 | 3 | 20 | 2 | 399 | 416 | N |
| | pp65 | | RK (92) | | | | | | | | | | | | |
| P06725\|PP65_HCMVA | tegument protein | UL83 | SDEELVTTERKTPR | 14 | Y | 86.45 | 1660 | 2.6 | 416 | 4 | 13 | 5 | 406 | 419 | N |
| | pp65 | | (93) | | | | | | | | | | | | |
| P06725\|PP65_HCMVA | tegument protein | UL83 | SDEELVTTER (94) | 10 | Y | 80.49 | 1178 | 6.1 | 590 | 2 | 15 | 4 | 406 | 415 | N |
| | pp65 | | | | | | | | | | | | | | |
| P06725\|PP65_HCMVA | tegument protein | UL83 | SDEELVTTERK (95) | 11 | N | 62.71 | 1306 | −5.2 | 436 | 3 | 12 | 3 | 406 | 416 | N |
| | pp65 | | | | | | | | | | | | | | |
| P06725\|PP65_HCMVA | tegument protein | UL83 | EELVTTERKTPR (96) | 12 | Y | 65.51 | 1458 | 1.8 | 365 | 4 | 11 | 2 | 408 | 419 | N |
| | pp65 | | | | | | | | | | | | | | |
| P06725\|PP65_HCMVA | tegument protein | UL83 | EELVTTER (97) | 8 | N | 50.32 | 975.5 | 1.6 | 489 | 2 | 13 | 2 | 408 | 415 | N |
| | 2055 | | | | | | | | | | | | | | |
| P06725\|PP65_HCMVA | tegument protein | UL83 | VTGGGAMAGASTSA | 16 | Y | 54.29 | 1350 | 2.7 | 676 | 2 | 12 | 2 | 420 | 435 | N |
| | pp65 | | GR (98) | | | | | | | | | | | | |
| P06725\|PP65_HCMVA | tegument protein | UL83 | SASSATACTSGVMTR | 15 | N | 71.02 | 1548 | 7.1 | 517 | 3 | 13 | 3 | 439 | 453 | Y |
| | pp65 | | (99) | | | | | | | | | | | | |
| P06725\|PP65_HCMVA | tegument protein | UL83 | SASSATACTSGVMT | 14 | Y | 67.79 | 1392 | −1.1 | 697 | 2 | 14 | 3 | 439 | 452 | Y |
| | pp65 | | (100) | | | | | | | | | | | | |
| P06725\|PP65_HCMVA | tegument protein | UL83 | GRLKAESTVAPEED | 14 | N | 68.86 | 1501 | 5.8 | 501 | 3 | 12 | 2 | 454 | 467 | N |
| | pp65 | | (101) | | | | | | | | | | | | |
| P06725\|PP65_HCMVA | tegument protein | UL83 | NLVPMIVATVQGQNL | 15 | Y | 75.62 | 1611 | 2.6 | 806 | 2 | 30 | 1 | 495 | 509 | N |
| | pp65 | | K (102) | | | | | | | | | | | | |
| P06725\|PP65_HCMVA | tegument protein | UL83 | NLVPMVATVQGQNL | 14 | N | 41.96 | 1499 | −12.3 | 750 | 2 | 32 | 3 | 495 | 508 | Y |
| | pp65 | | (103) | | | | | | | | | | | | |
| P06725\|PP65_HCMVA | tegument protein | UL83 | AELEGVWQPAAQPK | 14 | Y | 88.71 | 1523 | 3.7 | 762 | 2 | 26 | 9 | 525 | 538 | N |
| | pp65 | | (104) | | | | | | | | | | | | |
| P06725\|PP65_HCMVA | tegument protein | UL83 | EGVWQPAAQPK | 13 | Y | 78.64 | 1210 | −7.8 | 606 | 2 | 17 | 3 | 528 | 538 | N |
| | pp65 | | (105) | | | | | | | | | | | | |
| P06725\|PP65_HCMVA | tegument protein | UL83 | HRQDALPGPCIASTPK | 16 | Y | 75.59 | 1809 | 1.5 | 453 | 4 | 14 | 3 | 542 | 557 | Y |
| | pp65 | | (106) | | | | | | | | | | | | |
| P06725\|PP65_HCMVA | tegument protein | UL83 | QDALPGPCIASTPK | 14 | N | 47.13 | 1499 | 2.8 | 750 | 2 | 25 | 2 | 544 | 557 | Y |
| | pp65 | | (107) | | | | | | | | | | | | |
| P16727\|UL84_HCMVA | Protein UL84 | UL84 | TLGPSVFGRLELD | 13 | N | 43.09 | 1403 | −2.2 | 702 | 2 | 41 | 1 | 339 | 351 | N |
| | | | (108) | | | | | | | | | | | | |
| P16789\|AN_HCMVA | Alkaline nuclease | UL98 | LSRKTNLPIWVPNSAN | 29 | N | 81.95 | 3206 | 12.3 | 802 | 4 | 36 | 1 | 556 | 584 | N |
| | | | EYVVSSVPRPVSP | | | | | | | | | | | | |
| | | | (109) | | | | | | | | | | | | |

In some embodiments, additional viral candidate peptides for HLA-E ligands can be identified by searching sequences from other CMV strains obtainable from a database, e.g. GenBank that are similar to those identified in an assay, e.g. those from Table 1. Some of the similar sequences identified from other CMV strains are listed below.

TABLE 2
Sequences from other CMV strains deposited in GenBank that are similar to CMV
peptides isolated from HLA-E from AD169 strain CMV-infected cells

| Sequence identified from AD169 | |
|---|---|
| strain CMV-infected cells | Similar sequences from other CMV strains |
| UL22A | APSQKSKRSVTVEQPGTSADGSN (SEQ ID NO: 110) |
| APSQKSKRSVTVEQPSTSADGSN | AHJ86121.1 |
| (SEQ ID NO: 7) | APSQKSKRSVTVEQHSTSADGSN (SEQ ID NO: 111) |
| | AKI19951.1 |
| | APSQKSKRSVTVDQPNTSADGSN (SEQ ID NO: 112) |
| | AKI18612.1 |
| | APSQKSKRSVTVEQPSTSTNSDGNN (SEQ ID NO: 113) |
| | AKI08582.1 |
| | APSQKSKRSVTVEQPSTSTNSDG (SEQ ID NO: 114) |
| | AAL08513.1 |
| | APSQKSKRSVTVEQPSTSTNSGGN (SEQ ID NO: 115) |
| | AAL08517.1 |
| UL35 GEPREAQLDLEAD (SEQ ID | GEPRETQLDLEAD (SEQ ID NO: 116) AKI13612.1 |
| NO: 23) | GEPHEAQLDLEAD (SEQ ID NO: 117) AHJ85631.1 |
| UL35 RAPLGQESEPEITEHR (SEQ | RAPLGQGSEPEITEHR (SEQ ID NO: 118) |
| ID NO: 26) | AHJ84113.1 |
| UL35 PRDDLAENLRHL (SEQ ID | PRDDLAENLRNL (SEQ ID NO: 119) |
| NO: 27) | AKI24347.1 |
| UL37 PPAGSTSVSLPPASP (SEQ ID | PPAGSTSVSLPLASP (SEQ ID NO: 120) |
| NO: 28) | AKI24695.1 |
| UL42 TVVINRDSANITTGTQASSG | TVVINRDNTNITTGTQASTSG (SEQ ID NO: 121) |
| (SEQ ID NO: 32) | AFR54870.1 |
| | TVVINRDSANTTTGVSSSSG (SEQ ID NO: 122) |
| | ACZ79963.1 |
| | TVVINRDSANTTTGVSSASSG (SEQ ID NO: 123) |
| | AHJ83781.1 |
| | TVVINRDSSNTTTGT-PSSG (SEQ ID NO: 124) |
| | AHV83999.1 |
| | TVVINRDSSNTTTGRQ (SEQ ID NO: 125) |
| | AKI17961.1 |
| | TVVINRDNSN-TTGTVSTSG (SEQ ID NO: 126) |
| | AKI12281.1 |
| | TVVINRDN-STTTGT-SSG (SEQ ID NO: 127) |
| | AKI22307.1 |
| | TVVINRDN-STATGTASSSG (SEQ ID NO: 128) |
| | AKI07597.1 |
| UL44 PFDKNYVGNSGK (SEQ ID | PFDKNYVGNSSK (SEQ ID NO: 129) |
| NO: 36) | AHV84001.1 |

### Example 3

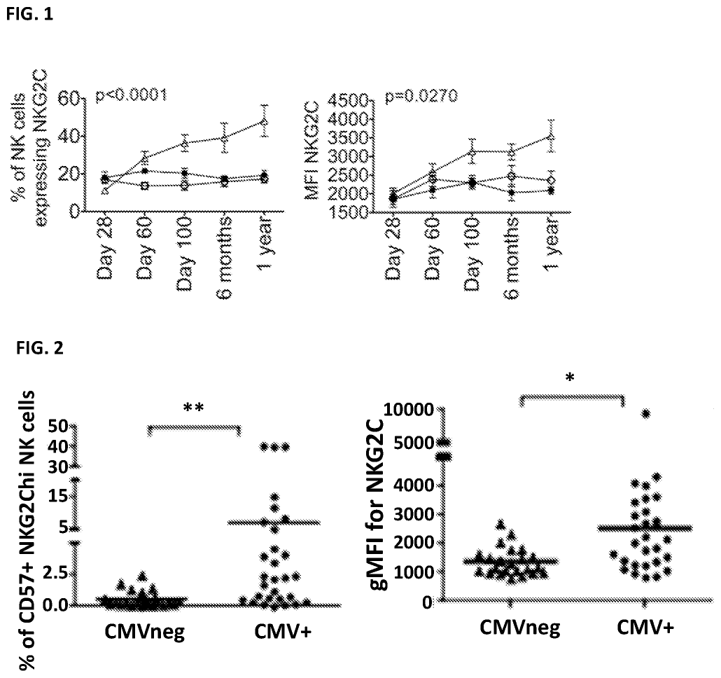

If a specific subset of human NK cells recognizes CMV these NK cells would preferentially expand during acute CMV infection and persist. By studying solid organ and hematopoietic stem cell transplant patients who reactivated CMV the inventors have shown that NK cells expressing CD94-NKG2C specifically respond. An increased number of NKG2C+ NK cells was observed in these patients more than a year after control of CMV (FIG. 1). In contrast, in healthy adults and transplant patients who did not reactivate CMV, the number and frequency of CD94-NKG2C⁺ NK cells remains stable, although the variation in the frequency of NKG2C+ in healthy individuals may reflect recent re-activation or re-infection. A dramatic expansion of NKG2C⁺ NK cells was observed in an infant with congenital T-B+NK+ SCID immunodeficiency infected with CMV. An increase in the cell surface density of NKG2C on NK cells from CMV-seropositive was observed as compared to CMV-seronegative individuals. This is consistent with the increased levels of the CMV-specific Ly49H receptor on mouse NK cells repetitively infected with CMV. Thus, although mouse Ly49H and human CD94-NKG2C receptors do not undergo somatic mutation, NK cells expressing higher amounts of these receptors might preferentially proliferate or survive after CMV infection.

The frequency of NKG2C+NK cells is dramatically increased in CMV-seropositive compared with CMV-seronegative healthy individuals, and this is observed in donors possessing both HLA-E*01:01 and HLA-E*01:03. It was also noted a specific expansion of NKG2C⁺ NK cells that co-express CD57, a marker of NK cell maturation, in CMV-seropositive adults (FIG. 2). Of note, the expansion of NKG2C⁺ NK cells is not observed in CMV-seronegative blood donors who were positive for other herpesviruses. In collaborative studies with Dr. Kristen Hogquist (Univ. Minn.) the inventors evaluated, in a longitudinal study, the NK cell response in individuals before, during, and after acute EBV infection (i.e. mononucleosis disease) and demonstrated that the NKG2C⁺ NK cells do not respond (Hendricks and Lanier), despite the induction of a robust EBV-specific CTL response. Increased frequencies of NKG2C⁺ NK cells have been detected after HIV-1, Hepatitis B and C, Chikungunya, and Hantavirus infection, but this only occurred in CMV-seropositive donors (i.e. individuals already infected with CMV) and never in CMV-seronegative subjects, suggesting that reactivation of CMV at subclinical levels might be triggered during these other infections. In vitro co-culture of peripheral blood lymphocytes from CMV-seropositive individuals with AD169, Towne, or Toledo strain CMV-infected fibroblasts results in the preferential expansion of NKG2C⁺ NK cells, demonstrating that all of these CMV strains are recognized by NKG2C⁺ NK cells. This was blocked using a neutralizing anti-CD94 mAb, thus directly implicating CD94-NKG2C in this CMV-specific response. NKG2C⁺ NK cells were not expanded when co-cultured with fibroblasts exposed to UV-inactivated CMV, showing that viral infection is required. Taken together, these data point to significant involvement of CD94-NKG2C in the response to CMV infection.

HLA-E/peptide ligands can be tested for binding to CD94-NKG2C and activation of NKG2C+ NK cells. There is a technology (e.g. by Dr. Hildebrand) to facilitate the purification and highly systematic characterization of the peptide repertoire of HLA class I molecules in virus-infected cells. This technique involves stable transfection of a selected cell line with the desired HLA class T molecule that has been truncated after the extracellular domain to generate a soluble HLA (sHLA) class I protein that is secreted into the culture supernatant. sHLA class I protein is transported through the normal antigen-processing pathway and naturally loaded with endogenous peptides prior to secretion of the soluble molecule. This method has been validated by transfection of cells (lung epithelial, T, cervical epithelial) with soluble HLA-A*02:01 or HLA-B*07:02 and infection with influenza A virus, West Nile virus, and HIV-1. sHLA proteins were isolated from the supernatant of infected or uninfected cells by immunoaffinity chromatography, peptides were acid eluted, separated by reverse-phase high-pressure liquid chromatography (RP-HPLC), and comparatively ion mapped by mass spectroscopy (MS1). Peptides unique to infected cells were then amino acid sequenced by tandem mass spectroscopy (MS2) fragmentation. Comparison of the peptide repertoire in the infected and uninfected cells identified not only naturally processed viral peptides, but host-derived peptides unique to infected cells were also detected due to the cellular stress caused by infection.

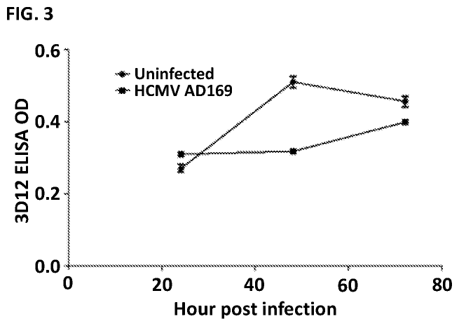

A soluble construct of HLA-E*01:03 was constructed by removing the transmembrane and cytoplasmic domains. An epitope tag, VLDL-r, was incorporated into the C-terminus of HLA-E*01:03, and the construct was cloned into the pcDNA3.1(-) vector and stably transfected into human U-373MG cells, which can be productively infected by CMV. Soluble sHLA-E*01:03 production was monitored using an ELISA with anti-VLDL-r and anti-HLA-E (mAb 3D12). Previously, the sHLA-E*01:03-transfected U-373MG cells were infected with AD169 strain CMV (for which there is a complete genomic sequence GenBank: BK000394.5, at an MOI of 3-5. Cells were confirmed as infected by morphological evaluation at 72 h post-infection and supernatants were collected at 24, 48, and 72 h. Soluble HLA-E was successfully harvested from CMV-infected cells as measured by ELISA (FIG. 3). The highest producing clone of the sHLA-E*01:03-transfected U-373MG cells was expanded into 2-liter roller bottles and 10 billion cells were seeded into 30 kDa cut-off hollow-fiber bioreactors for large-scale sHLA-E*01:03 production. The preliminary data presented in FIG. 3 demonstrate that plentiful sHLA-E*01:03 is secreted by U-373MG transfectants after infection with CMV and sHLA-E*01:03-transfected U-373MG cells yield peptide ligand that may be sufficient for comparative MS analysis.

In some embodiments, the candidate peptides can be tested by culturing the CMV peptides at a range of concentrations with peripheral blood mononuclear cells from CMV-seropositive and CMV-seronegative individuals and then measuring their production of interferon-gamma by T cells by well-established techniques, such as ELISPOT, ELISA, or intracellular staining methods. The procedure can be conducted in the presence or absence of a blocking antibody reactive with HLA-E to determine whether the peptides are being presented by HLA-E polypeptides on antigen-presenting cells. Fluorescent tetrameric complexes constructed with the candidate CMV peptides bound to HLA-E, prepared by standard methods, can be used to stain T cells or NK cells from CMV-seropositive and CMV-seronegative individuals to identify NK cells or T cells specifically binding to the HLA-E-CMV peptide complexes. Blocking antibodies against NKG2C or the T cell receptor can be used to determine if the complexes are reacting with the CD94-NKG2C or T cell receptors on the NK cells or T cells. These HLA-E-CMV peptide tetramers can also be used to monitor expansion of NK cells or T cells in patients receiving a CMV vaccine.

While preferred embodiments of the disclosures have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### SEQUENCE LISTING

| SEQ ID | | |
|---|---|---|
| NO | Sequence | Description |
| 1 | MVDGTLLLLLSEALALTQTWAGSHSLKYFHTSVSRPGRGEPRFISVGYVDDT | HLA-E*01:01 |
| | QFVRFDNDAASPRMVPRAPWMEQEGSEYWDRETRSARDTAQIFRVNLRTLR | |
| | RYYNQSEAGSHTLOWMHGCELGPDRRFLRGYEQFAYDGKDYLTLNEDLRS | |
| | WTAVDTAAQISEQKSNDASEAEHQRAYLEDTCVEWLHKYLEKGKETLLHLE | |
| | PPKTHVTHHPISDHEATLRCWALGFYPAEITLTWQQDGEGHTQDTELVETRP | |
| | AGDGTFQKWAAVVVPSGEEQRYTCHVQHEGLPEPVTLRWKPASQPTIPIVGI | |
| | TAGLVLLGSVVSGAVVAAVIWRKKSSGGKGGSYSKAEWSDSAQGSESHSL | |
| 2 | MVDGTLLLLLSEALALTQTWAGSHSLKYFHTSVSRPGRGEPRFISVGYVDDT | HLA-E*01:03 |
| | QFVRFDNDAASPRMVPRAPWMEQEGSEYWDRETRSARDTAQIFRVNLRTLR | |
| | GYYNQSEAGSHTLQWMHGCELGPDGRFLRGYEQFAYDGKDYLTLNEDLRS | |
| | WTAVDTAAQISEQKSNDASEAEHQRAYLEDTCVEWLHKYLEKGKETLLHLE | |
| | PPKTHVTHHPISDHEATLRCWALGFYPAEITLTWQQDGEGHTQDTELVETRP | |
| | AGDGTFQKWAAVVVPSGEEQRYTCHVQHEGLPEPVTLRWKPASQPTIPIVGI | |
| | IAGLVLLGSVVSGAVVAAVIWRKKSSGGKGGSYSKAEWSDSAQGSESHSL | |
| 3 | MVDGTLLLLLSEALALTQTWAGSHSLKYFHTSVSRPGRGEPRFISVGYVDDT | SHLA-E*0103 |
| | QFVRFDNDAASPRMVPRAPWMEQEGSEYWDRETRSARDTAQIFRVNLRTLR | tVLDLr |
| | GYYNQSEAGSHTLQWMHGCELGPDRRFLRGYEQFAYDGKDYLTLNEDLRS | |
| | WTAVDTAAQISEQKSNDASEAEHQRAYLEDTCVEWLHKYLEKGKETLLHLE | |
| | PPKTHVTHHPISDHEATLRCWALGFYPAEITLTWQQDGEGHTQDTELVETRP | |
| | AGDGTFQKWAAVVVPSGEEQRYTCHVQHEGLPEPVTLRWSVVSTDDDLA | |
| 4 | PTYDELPSRPPQ | IRS1 peptide |
| 5 | LDIVEEDEWLR | UL13 peptide |
| 6 | APSQKSKRSVTVEQPSTSAD | UL22A peptide |
| 7 | APSQKSKRSVTVEQPSTSADGSN | UL22A peptide |
| 8 | SQKSKRSVTVEQPSTSAD | UL22A peptide |
| 9 | RSVTVEQPSTSAD | UL22A peptide |
| 10 | SVTVEQPSTSAD | UL22A peptide |
| 11 | SVTVEQPSTSADGSN | UL22A peptide |
| 12 | SVTVEQPSTSA | UL22A peptide |
| 13 | VTVEQPSTSAD | UL22A peptide |
| 14 | GDEDYSGEYDVL | UL22A peptide |
| 15 | LITDGDGSEIIQQP | UL22A peptide |
| 16 | EHKENQAKENEKKIQ | UL22A peptide |
| 17 | SSVSGVRTPR | UL31 peptide |
| 18 | SLQFIGLQRRD | UL32 peptide |
| 19 | TVAFDLSSPQK | UL32 peptide |
| 20 | AQGSRAPSGPPLPVLPVD | UL35 peptide |
| 21 | ARERGEFGDEDEEQENDGEPR | UL35 peptide |
| 22 | ARERGEFGDEDEEQEND | UL35 peptide |
| 23 | GEPREAQLDLEAD | UL35 peptide |
| 24 | EAQLDLEAD | UL35 peptide |
| 25 | RAPLGQESEPEITEH | UL35 peptide |
| 26 | RAPLGQESEPEITEHR | UL35 peptide |
| 27 | PRDDLAENLRHL | UL35 peptide |
| 28 | PPAGSTSVSLPPASP | UL37 peptide |
| 29 | VMAPRTLIL | UL40 peptide |
| 30 | VMAPRTLI | UL40 peptide |
| 31 | VMAPRTL | UL40 peptide |
| 32 | TVVINRDSANITTGTQASSG | UL42 peptide |
| 33 | TINNSTPLL | UL44 peptide |
| 34 | FGVVADLLKWIGPHTRV | UL44 peptide |
| 35 | PFDKNYVGNSGKSRGGGGGGGSLSSLANAGGLHD | UL44 peptide |
| 36 | PFDKNYVGNSGK | UL44 peptide |
| 37 | PFDKNYVGNSG | UL44 peptide |
| 38 | PFDKNYVGNSGKSRGGGGGGGSLSSLANAGGLHDD | UL44 peptide |
| 39 | PFDKNYVGNSGKS | UL44 peptide |
| 40 | PFDKNYVGNSGKSRGGGGGGGSLSSLANA | UL44 peptide |
| 41 | PFDKNYVGNSGKSRGGGGGGGSLSSL | UL44 peptide |
| 42 | SRGGGGGGGSLSSLANAGGLHDD | UL44 peptide |
| 43 | GPGLDNDLMNEPMGLGGLGGGGGGGGKKH | UL44 peptide |
| 44 | SEDSVTFEFVPNTKKQ | UL44 peptide |
| 45 | SVTFEFVPNTK | UL44 peptide |
| 46 | VTFEFVPNTK | UL44 peptide |
| 47 | MDARAVAKRPRD | UL46 peptide |
| 48 | NFSVELGDFREFV | UL46 peptide |
| 49 | SNTAPGPTVANKRD | UL48A peptide |
| 50 | SNTAPGPTVAN | UL48A peptide |
| 51 | PTYVIDKYV | UL52 peptide |
| 52 | PVTGEDTFSAHGKSD | UL57 peptide |
| 53 | QNVALITAT | UL57 peptide |
| 54 | EAGGVGGSSGGGGGSGLLPAKRSRL | UL57 peptide |
| 55 | EAGGVGGSSGGGGGSGLLPAKRS | UL57 peptide |
| 56 | EAGGVGGSSGGGGGSGLLPAKR | UL57 peptide |
| 57 | PATLTAYDK | UL69 peptide |
| 58 | QPPPPPPPP | UL69 peptide |
| 59 | EQDRLLVDL | UL82 peptide |
| 60 | PNTYIHKTETD | UL82 peptide |
| 61 | DEDDLSSTPTPTPL | UL82 peptide |
| 62 | LSSTPTPTPL | UL82 peptide |
| 63 | MDGDVRTAADISSTLR | UL82 peptide |
| 64 | GDVRTAADISSTLRSVPAPRPSPISTASTSSTPR | UL82 peptide |
| 65 | GDVRTAADISSTLRSVPAPRPSPISTA | UL82 peptide |
| 66 | VRTAADISSTLRSVPAPRPSP | UL82 peptide |
| 67 | VRTAADISSTLR | UL82 peptide |
| 68 | VRTAADISSTLRSVPAPRPSPISTA | UL82 peptide |
| 69 | ISSTLRSVPAPRPSPISTA | UL82 peptide |
| 70 | SVPAPRPSPISTASTSSTPR | UL82 peptide |
| 71 | SVPAPRPSPISTA | UL82 peptide |
| 72 | SVPAPRPSPISTAST | UL82 peptide |
| 73 | SVPAPRPSPIST | UL82 peptide |
| 74 | MISVLGPISGHVLKAVFSRGD | UL83 peptide |
| 75 | MISVLGPISGHVLK | UL83 peptide |
| 76 | SVLGPISGIIVLK | UL83 peptide |
| 77 | SVLGPISGHV | UL83 peptide |
| 78 | VLGPISGHV | UL83 peptide |
| 79 | VLGPISGHVLK | UL83 peptide |
| 80 | SEVENVSVNVHNPTGR | UL83 peptide |
| 81 | SEVENVSVNVHNPTG | UL83 peptide |
| 82 | HRHLPVAD | UL83 peptide |
| 83 | TSAFVFPTK | UL83 peptide |
| 84 | WDRIIDEGAAQGDDDVWTSGSDSDEELVTTER | UL83 peptide |
| 85 | AQGDDDVWTSGSDSDEELVTTER | UL83 peptide |
| 86 | GDDDVWTSGSDSDEELVTTER | UL83 peptide |
| 87 | DDDVWTSGSDSDEELVTTER | UL83 peptide |
| 88 | DVWTSGSDSDEELVTTER | UL83 peptide |
| 89 | VWTSGSDSDEELVTTER | UL83 peptide |
| 90 | VWTSGSDSDEELVTTERKTPR | UL83 peptide |
| 91 | VWTSGSDSDEELVTTE | UL83 peptide |
| 92 | VWTSGSDSDEELVTTERR | UL83 peptide |
| 93 | SDEELVTTERKTPR | UL83 peptide |
| 94 | SDEELVTTER | UL83 peptide |
| 95 | SDEELVTTERK | UL83 peptide |
| 96 | EELVTTERKTPR | UL83 peptide |
| 97 | EELVTTER | UL83 peptide |
| 98 | VTGGGAMAGASTSAGR | UL83 peptide |
| 99 | SASSATACTSGVMTR | UL83 peptide |
| 100 | SASSATACTSGVMT | UL83 peptide |
| 101 | GRLKAESTVAPEED | UL83 peptide |
| 102 | NLVPMVATVQGQNLK | UL83 peptide |
| 103 | NLVPMVATVQGQNL | UL83 peptide |
| 104 | AELEGVWQPAAQPK | UL83 peptide |
| 105 | EGVWQPAAQPK | UL83 peptide |
| 106 | HRQDALPGPCIASTPK | UL83 peptide |
| 107 | QDALPGPCIASTPK | UL83 peptide |
| 108 | TLGPSVFGRLELD | UL84 peptide |
| 109 | LSRKTNLPIWVPNSANEYVVSSVPRPVSP | UL98 peptide |
| 110 | APSQKSKRSVTVEQPGTSADGSN | AHJ86121.1 peptide |
| 111 | APSQKSKRSVTVEQHSTSADGSN | AKI19951.1 peptide |
| 112 | APSQKSKRSVTVDQPNTSADGSN | AKI18612.1 peptide |
| 113 | APSQKSKRSVTVEQPSTSTNSDGNN | AKI08582.1 peptide |
| 114 | APSQKSKRSVTVEQPSTSTNSDG | AAL08513.1 peptide |
| 115 | APSQKSKRSVTVEQPSTSTNSGGN | AAL08517.1 peptide |
| 116 | GEPRETQLDLEAD | AKI13612.1 peptide |
| 117 | GEPHEAQLDLEAD | AHJ85631.1 peptide |
| 118 | RAPLGQGSEPEITEIIR | AIIJ84113.1 peptide |
| 119 | PRDDLAENLRNL | AKI24347.1 peptide |
| 120 | PPAGSTSVSLPLASP | AKI24695.1 peptide |
| 121 | TVVINRDNTNITTGTQASTSG | AFR54870.1 peptide |
| 122 | TVVINRDSANTTTGVSSSSG | ACZ79963.1 peptide |
| 123 | TVVINRDSANTTTGVSSASSG | AHJ83781.1 peptide |
| 124 | TVVINRDSSNTTTGTPSSG | AHV83999.1 peptide |
| 125 | TVVINRDSSNTTTGRQ | AKI17961.1 peptide |
| 126 | TVVINRDNSNTTGTVSTSG | AKI12281.1 peptide |
| 127 | TVVINRDNSTTTGTSSG | AKI22307.1 peptide |
| 128 | TVVINRDNSTATGTASSSG | AKI07597.1 peptide |
| 129 | PFDKNYVGNSSK | AHV84001.1 peptide |

1. A composition comprising:
at least one human leukocyte antigen E (HLA-E) ligand or a functional fragment, derivative or variant thereof capable of binding to HLA-E, wherein the HLA-E ligand or the functional fragment, derivative or variant thereof comprises an amino acid sequence having at least 85% identity to a sequence selected from the group consisting of SEQ ID NOs: 4-28 and 32-129; and
a pharmaceutically acceptable carrier,
wherein the composition is capable of inducing an immune response.

2. The composition of claim 1, wherein the HLA-E ligand is capable of binding to a) a CD94-NKG2C receptor that is present on natural killer (NK) cells or T cells; or b) a T cell antigen receptor.

3-4. (canceled)

5. The composition of claim 1, further comprising a linker.

6. The composition of claim 1, wherein the composition comprises more than one HLA-E ligand or a functional fragment, derivative or variant thereof.

7. The composition of claim 1, further comprising:
HLA-E or a functional fragment, derivative or variant thereof.

8. The composition of claim 7, wherein the HLA-E or the functional fragment, derivative or variant thereof is a soluble and secretory form.

9. (canceled)

10. The composition of claim 1, wherein the immune response comprises proliferation of T cells and/or NK cells.

11-12. (canceled)

13. The composition of claim 1, wherein the immune response comprises increase of T cells and/or NK cells that express CD94-NKG2C receptor.

14. The composition of claim 1, wherein the immune response comprises killing of CMV-infected cells.

15-18. (canceled)

19. The composition of claim 7, wherein the HLA-E or the functional fragment, derivative or variant of HLA-E and the HLA-E ligand or the functional fragment, derivative or variant of HLA-E ligand do not bind to inhibitory CD94-NKG2A receptor of NK cells or T cells.

20. The composition of claim 1, wherein the composition is formulated as a vaccine.

21. The composition of claim 1, wherein the HLA-E ligand or the functional fragment, derivative or variant thereof is comprises an amino acid sequence selected from the group consisting of the sequences of SEQ ID NOs: 4-28 and 32-129.

22. The composition of claim 7, wherein the HLA-E or the fragment, derivative or variant thereof comprises the sequence of SEQ ID NO. 1, 2, or 3, or a variant thereof having at least 85% identity to the sequence of SEQ ID NO. 1, 2, or 3.

23. The composition of claim 7, wherein the HLA-E or the functional fragment, derivative or variant of HLA-E and the HLA-E ligand or the functional fragment, derivative or variant of HLA-E ligand are covalently associated via a linker

24. The composition of claim 23, wherein the linker comprises the sequence of a (G₄S)₃, (G₄S)₄ or (G₄S)₅.

25. A method of inducing an immune response in a subject, the method comprising:
administering to the subject an effective amount of a composition comprising:
at least one HLA-E ligand or a functional fragment, derivative or variant thereof capable of binding to HLA-E, wherein the HLA-E ligand or the functional fragment, derivative or variant thereof comprises an amino acid sequence having at least 85% identity to a sequence selected from the group consisting of SEQ ID NOs: 4-28 and 32-129; and
a pharmaceutically acceptable carrier.

26-35. (canceled)

36. A method of making a composition for inducing an immune response, the method comprising:
formulating at least one HLA-E ligand or a functional fragment, derivative or variant thereof capable of binding to HLA-E, wherein the HLA-E ligand or the functional fragment, derivative or variant thereof comprises an amino acid sequence having at least 85% identity to a sequence selected from the group consisting of SEQ ID NOs: 4-28 and 32-129 and a pharmaceutically acceptable carrier in a form suitable for administration.

37. (canceled)

38. A method of making a composition for inducing an immune response, the method comprising:
introducing a vector sequence encoding a recombinant protein to mammalian cells, allowing expression of the recombinant protein, wherein the recombinant protein comprises at least one HLA-E ligand or a functional fragment, derivative or variant thereof capable of binding to HLA-E, wherein the HLA-E ligand or the functional fragment, derivative or variant thereof comprises an amino acid sequence having at least 85% identity to a sequence selected from the group consisting of SEQ ID NOs: 4-28 and 32-129;
isolating the expressed recombinant protein; and
formulating the isolated recombinant protein and a pharmaceutically acceptable carrier in a form suitable for administration.

39-42. (canceled)
